(19) United States
(12) Patent Application Publication
(10) Pub. No.: US 2011/0065774 A1
(43) Pub. Date: Mar. 17, 2011

(54) CHEMICALLY MODIFIED OLIGONUCLEOTIDES AND USES THEREOF

(75) Inventor: Muthiah Manoharan, Cambridge, MA (US)

(73) Assignee: ALNYLAM PHARMACEUTICALS, Cambridge, MA (US)

(21) Appl. No.: 12/865,636

(22) PCT Filed: Jan. 30, 2009
(86) PCT No.: PCT/US2009/032635

§ 371 Date: Nov. 30, 2010

Related U.S. Application Data
(60) Provisional application No. 61/025,130, filed on Jan. 31, 2008.

(51) Int. Cl. A61K 31/713; C07H 21/02; A61P 43/00; A61P 27/02

(52) U.S. Cl. 514/44 A; 536/24.5

(57) ABSTRACT

This invention relates generally to chemically oligonucleotides (e.g., modified oligonucleotides) useful for augmenting activity of a target gene.

### PRIORITY CLAIM

This invention claims benefit under 35 U.S.C. §371 of PCT Application No. PCT/US09/032,635, filed Jan. 30, 2009, which claims priority to U.S. Provisional Application No. 61/025,130, filed Jan. 31, 2008, both of which are herein incorporated by reference in their entirety.

### TECHNICAL FIELD

This invention relates generally to agents, e.g., chemically modified oligonucleotides useful for increasing the expression of a gene product in a cell. More particularly, the invention relates to a small activating ribonucleotide structure (“saRNA”) that is complementary to a non-coding nucleic acid sequence of a gene.

### BACKGROUND

A variety of nucleic acid species are capable of modifying gene expression. These include antisense RNA, siRNA, microRNA, RNA and DNA aptamers, and decoy RNAs. Each of these nucleic acid species can inhibit target nucleic acid activity, including gene expression.

saRNAs are a class of oligonucleotide agents that are complementary to a non-coding strand of a gene sequence. saRNAs can increase expression of a gene target when contacted with a call. Examples of oligonucleotide agents that increase gene expression include those described in WO 2006/113246 and WO 2007/086990.

### SUMMARY

The present invention is based in part on the discovery that oligonucleotide agents that modulate gene expression (e.g., increase or decrease gene expression), e.g., saRNAs, can be modified, for example, chemically modified, to provide improved properties, such as improved stability and/or resistances to degrading enzymes.

An oligonucleotide agent featured herein for example an oligonucleotide agent that modulates gene expression by either increase or decreasing gene expression, e.g., an saRNA, is modified, for example chemically modified. In some instances the modified agent (e.g., saRNA) has increased stability against nucleolytic degradation. Exemplary modifications include a modification of the nucleotide backbone such as modification of the phosphate linker or replacement of the phosphate linker; modification of the sugar moiety such as modification of the 2′ hydroxyl on the ribose; replacement of the sugar moiety such as ribose or deoxyribose with a different chemical structure such as a PNA structure; or modification of the nucleobase for example modification to a universal base or G-clamp. In some embodiments, the oligonucleotide agent includes a phosphorothioate in at least the first, second, or third internucleotide linkage at the 5′ or 3′ end of the nucleotide sequence. In one embodiment, the oligonucleotide agent includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In a particularly preferred embodiment, the oligonucleotide agent includes at least one 2′-O-methyl-modified nucleotide, and in some embodiments, all of the nucleotides of the oligonucleotide agent include a 2′-O-methyl modification.

The oligonucleotide agent can be further modified so as to be attached to a ligand, for example, a ligand selected to improve stability, distribution or cellular uptake of the agent, e.g., cholesterol or folate. Exemplary lipophilic ligands include a cholesterol; a bile acid; and a fatty acid (e.g., lithocholic-oleyl, lauroyl, docosnyl, stearoyl, palmitoyl, myristoyl, oleoyl, linoleoyl). In some preferred embodiments, the oligonucleotide agent is combined with a targeting agent such as a folate moiety.

The oligonucleotide agent can further be in isolated form or can be part of a a delivery vehicle or pharmaceutical composition used for the methods described herein, particularly as a pharmaceutical composition formulated for parental administration. The pharmaceutical compositions can contain one or more oligonucleotide agents, and in some embodiments, will contain two or more oligonucleotide agents, each one directed to a different non-coding sequence of a target gene.

In some embodiments, the expression of a gene can be modulated (e.g., increased or decrease) by an agent such as an oligonucleotide agent, including an saRNA e.g., through systemic administration of the agent, as well as by parenteral administration of such agents. Embodiments of the invention provide specific compositions and methods that are useful in modulating (e.g., augmenting) the expression of a gene, in e.g., a mammal, such as a human. In some embodiments, the oligonucleotide agent selectively increases synthesis of a target transcript on a gene. Exemplary genes for targeted for increased expression include b-globin (b-thallssemia), Dystrophin (Duchenne muscular dystrophy DMD), Cystic Fibrosis (Cystic Fibrosis Transmembrane Conductance Regulator CFTR), Tau, Lamin A (Cardiomyopathies, Peripheral Neuropathy, premature ageing syndrome), SMN1 (Spinal Muscular Atrophy), and OA1 (Ocular Albinism). Exemplary genes for modulation include bcl-xS, WT1 (Wilms' tumor suppressor), CD40, MyD88, and AChE.

An oligonucleotide agent, e.g., an saRNA, can be delivered to a cell or a human to augment transcription of a target gene, or activity of a target gene that is linked to a disease or disorder.

In one aspect, the invention features a method of modulating (e.g., augmenting or decreasing) the transcription of a target gene in a cell of a subject, e.g., a human subject. The method includes the step of administering an oligonucleotide agent, e.g., saRNA, to the subject, where the oligonucleotide agent is substantially single-stranded and includes a sequence that is substantially complementary to 12 to 23 contiguous nucleotides, and preferably 15 to 23 contiguous nucleotides, of non-coding region of a target gene (e.g., a promoter region). Such methods include contacting the cell with an oligonucleotide agent described herein for a time sufficient to allow uptake of the oligonucleotide agent into the cell. Such methods can be performed on a mammalian subject by administering to a subject one of the oligonucleotide agents/pharmaceutical compositions described herein.

In another aspect, the invention features a pharmaceutical composition including an oligonucleotide agent described herein, and a pharmaceutically acceptable carrier and/or delivery vehicle. In a preferred embodiment, the delivery vehicle is an association complex such as a liposome. A liposome generally includes a plurality of components such as one or more of a cationic lipid (e.g., an amino lipid), a targeting moiety, a fusogenic lipid, a PEGylated lipid. In some embodiments, the PEG-lipid is a targeted PEG-lipid. For example, a liposome can include a nucleic acid and an amine-lipid and a PEGylated lipid. In some embodiments, the PEG-lipid is a targeted PEG-lipid. In some embodiments, the preparation also includes a structural moiety such as cholesterol. Some preferred cationic lipids include, TETA-5LAP and DLinDMA. In some embodiments, the PEG lipid includes the following structure:

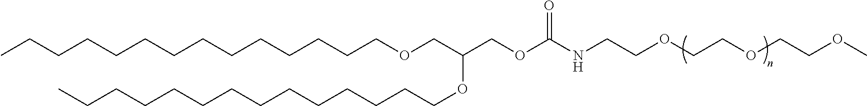

wherein the repeating PEG moiety has an average molecular weight of 2000 with n value between 42 and 47.

In some embodiments, the delivery vehicle includes a targeting agent such as galactose and Gal-NAc conjugates and clusters, folate, or transferrin. In some embodiments, the delivery vehicle includes a polymer or cyclodextran.

In another aspect, the invention provides methods of modulating (e.g., increasing or decreasing) expression of a target gene by providing an oligonucleotide agent to which a lipophilic moiety is conjugated, e.g., a lipophilic conjugated oligonucleotide agent described herein, to a cell. In some preferred embodiments, the method include increasing expression of a target gene. In certain preferred embodiments, the lipophilic moiety is a bile acid or a fatty acid (e.g., lithocholic-oleyl, lauroyl, docosnyl, stearoyl, palmitoyl, myristoyl, oleoyl, linoleoyl). In some embodiments, the oligonucleotide agent described herein is conjugated to a folate moiety. In a preferred embodiment the conjugated oligonucleotide agent can be used to modulate (e.g., increase) expression of a target gene in an organism, e.g., a mammal, e.g., a human, or to modulate (e.g., increase) expression of a target gene in a cell line or in cells which are outside an organism. In the case of a whole organism, the method can be used to increase expression of a gene and treat a condition associated with underexpression of the gene.

### DETAILED DESCRIPTION

The present invention is based in part on the discovery that activity levels of genes can be modulated (e.g., augmented) by an oligonucleotide agent described herein such as a modified saRNA, e.g., through systemic administration of the oligonucleotide agent, as well as by parenteral administration of such agents. In particular, the present invention provides specific compositions and methods that are useful for increasing expression levels of a target gene.

In one aspect, the invention features saRNA, for example, modified saRNAs. As used herein the term “saRNA” and the phrase “short activating RNA” refer to a ribonucleic acid molecule capable of facilitating gene activation and can be composed of a first ribonucleic acid strand comprising a ribonucleotide sequence complementary to a non-coding nucleic acid sequence of a gene and a second ribonucleic acid strand comprising a nucleotide sequence complementary to the first strand, wherein the first and second strands form a duplex region. The saRNA can also be composed of as a single strand RNA molecule that forms a double-stranded region, wherein the first region comprising a ribonucleotide sequence complementary to a non-coding nucleic acid sequence of the gene, and a second region comprising a ribonucleotide sequence complementary to the first region and forming a duplex region with the first region. The duplex region of an saRNA agent is usually between about 10 and about 50 base pairs in length, about 12 and about 48 base pairs, about 14 and about 46 base pairs, about 16 and about 44 base pairs, about 18 and about 42 base pairs, about 20 and about 40 base pairs, about 22 and about 38 base pairs, about 24 and about 36 base pairs, about 26 and about 34 base pairs, about 28 and about 32 base pairs, normally about 10, about, 15, about 20, about 25, about 30, about 35, about 40, about 45, about 50 base pairs in length. Additionally, the term saRNA and the phrase “small activating RNA” include nucleic acids that also contain moieties other than ribonucleotide moieties, including, but not limited to, modified nucleotides, modified internucleotide linkages, non-nucleotides, deoxynucleotides and analogs of the aforementioned nucleotides.

As used herein, the terms “gene activating”, “activating a gene”, or “gene activation” are interchangeable and refer to increasing gene expression with respect to transcription as measured by transcription level, niRNA level, enzymatic activity, methylation state, chromatin state or configuration, or other measure of its activity or state in a cell or biological system. Furthermore, “gene activating”, “activating a gene”, or “gene activation” refer to the increase of activity known to be associated with a nucleic acid sequence, such as its ability to function as a regulatory sequence, its ability to be transcribed, its ability to be translated and result in expression of a protein, regardless of the mechanism whereby such activation occurs.

saRNAs compounds described herein can be duplexes, and can be include separate strands or can be comprised of a single strand of RNA that forms short hairpin RNAs, RNAs with loops as long as, for example, about 4 to about 23 or more nucleotides, about 5 to about 22, about 6 to about 21, about 7 to about 20, about 8 to about 19, about 9 to about 18, about 10 to about 17, about 11 to about 16, about 12 to about 15, about 13 to about 14 nucleotides, RNAs with stem loop bulges, and short temporal RNAs. RNAs having loops or hairpin loops can include structures where the loops are connected to the stem by linkers such as flexible linkers. Flexible linkers can be selected of a wide variety of chemical structures, as long as they are of sufficient length and materials to enable effective intramolecular hybridization of the stem elements. Typically, the length to be spanned is at least about 10-24 atoms. In some embodiments, the saRNA agents are separate strands, e.g., two different strands that are not covalently linked.

The saRNA agents described herein generally include a region of complementarity to a non-coding region of appropriate length to provide for transcriptional activation of an adjacent coding sequence. saRNA agents also typically include 3′ terminal nucleotides which are not complementary to the non-coding sequence. saRNA typically comprise more than 10 nucleotides and less than 50 nucleotides, usually more than about 12 nucleotides and less than 48 nucleotides in length, such as about 14 nucleotides to about 46 nucleotides in length, about 16 nucleotides to about 44 nucleotides in length, including about 18 nucleotides to about 42 nucleotides in length, about 20 nucleotides to about 40 nucleotides in length, about 22 nucleotides to about 38 nucleotides in length, about 24 nucleotides to about 36 nucleotides in length, about 26 nucleotides to about 34 nucleotides in length, about 28 nucleotides to about 32 nucleotides in length. In representative embodiments, the saRNA agents comprise of about 14 nucleotides to about 30 nucleotides in length, such as about 15 nucleotides to about 29 nucleotides in length, about 16 nucleotides to about 28 nucleotides in length, including about 17 nucleotides to about 27 nucleotides in length, about 18 nucleotides to about 26 nucleotides in length, about 19 nucleotides to about 25 nucleotides in length, about 20 nucleotides to about 24 nucleotides in length, about 21 nucleotides to about 23 nucleotides in length, or about 22 nucleotides in length.

The saRNA agents described herein can be in double-stranded form, when in double stranded-form the saRNA agents typically comprises a region of complementarity greater than about 10 base pairs and less than about 50 base pairs in length. In some embodiments, the saRNA agents comprise a duplex region of between about 12 base pairs to about 48 base pairs in length, such as about 14 base pairs to about 46 base pairs in length, about 16 base pairs to about 44 base pairs in length, including about 18 base pairs to about 42 base pairs in length, about 20 base pairs to about 40 base pairs in length, about 22 base pairs to about 38 base pairs in length, about 24 base pairs to about 36 base pairs in length, about 26 base pairs to about 34 base pairs in length, about 28 base pairs to about 32 base pairs in length. In representative embodiments, the saRNA agents comprise of a duplex region between about 15 base pairs to about 30 base pairs in length, such as about 16 base pairs to about 29 base pairs in length, about 17 base pairs to about 28 base pairs in length, including about 18 base pairs to about 27 base pairs in length, about 19 base pairs to about 26 base pairs in length, about 20 base pairs to about 25 base pairs in length, about 21 base pairs to about 24 base pairs in length, about 22 base pairs to about 23 base pairs in length.

In some embodiments, the saRNA agents comprise a strand that is complementary to a portion of a non-coding nucleic acid sequence or a gene, e.g., a regulatory sequence, such as a promoter. In some embodiments, the strand is 100% complementary to the non-coding nucleic acid sequence of the gene, including about 99% complementary, 98% complementary, 97% complementary, 96% complementary, 95% complementary, 94% complementary, 93% complementary, 92% complementary, 91% complementary, 90% complementary, 85% complementary, 80% complementary, 75% complementary, 70% complementary to the non-coding nucleic acid sequence of the gene.

When designing the complementary strand of the saRNA agents described herein (e.g., the strand of the saRNA agent that is complementary to a portion of a non-coding nucleic acid sequence or a gene), the sequence is generally selected so as to avoid complementarity to any CpG island regions. By “CpG island region” is meant any region of the nucleic acid that is rich in the dinucleotide “CG” (Cytosine-Guanine).

In addition, when designing the complementary strand of the saRNA agents described herein (e.g., the strand of the saRNA agent that is complementary to a portion of a non-coding nucleic acid sequence or a gene), the sequence can be selected so as to avoid complementarity to any GC-rich regions. By “GC-rich” is meant any region of the nucleic acid that includes a greater number of guanine and cytosine base pairs compared to thymine and adenine base pairs as compared to the average number of guanine and cytosine residues in the rest of the genome in which the nucleic acid is present.

In some embodiments, the saRNA agents described herein are designed to avoid a non-coding nucleic acid sequence of a gene comprising a GC content greater than about 50% or less than about 30%. In certain embodiments, the saRNA agents of the subject invention will be designed in order to comprise a GC content greater than about 30% or less than about 50%, including a GC content of about 32%, a GC content of about 34%, a GC content of about 36%, a GC content of about 38%, a GC content of about 40%, a GC content of about 42%, a GC content of about 44%, a GC content of about 46%, a GC content of about 48%, a GC content of about 50%.

In some embodiments, the saRNA agents described herein are designed to comprise an AT content greater than about 50% to less than about 80%. In certain embodiments, the saRNA agents of the subject invention will be designed in order to comprise an AT content of about 52%, an AT content of about 54%, an AT content of about 56%, an AT content of about 58%, an AT content of about 60%, an AT content of about 62%, an AT content of about 64%, an AT content of about 66%, an AT content of about 68%, an AT content of about 70%, an AT content of about 72%, an AT content of about 74%, an AT content of about 76%, an AT content of about 78%, an AT content of about 80%.

In some embodiments, the saRNA agents described herein are designed to avoid a non-coding nucleic acid sequence of a gene comprising nucleotide repeats and low complex sequences, such as a sequence of four or more of the same base in a row, such as for example AAAA or CCCC. Moreover, the saRNA agents described herein will typically be designed in order to avoid a non-coding nucleic acid sequence of a gene comprising single nucleotide polymorphism (SNP) sites.

The saRNA agents described herein include a region of complementarity to non-coding target nucleic acid sequence. A non-coding target nucleic acid sequence refers to a nucleic acid sequence of interest that is not contained within an exon or is a regulatory sequence. In general, such a non-coding target sequence is a nucleic acid sequence approximately 2 kb upstream from the transcriptional start site of the target gene, including up to about 1.9 kb, about 1.8 kb, about 1.7 kb, about 1.6 kb, about 1.5 kb, about 1.4 kb, about 1.3 kb, about 1.2 kb, about 1.1 kb, about 1 kb, about 950 bp, about 900 bp, about 850 bp, about 800 bp, about 750 bp, about 700 bp, about 650 bp, about 600 bp, about 550 bp, about 500 bp, about 450 bp, about 400 bp, about 350 bp, about 300 bp, about 250 bp, about 200 bp, about 150 bp, about 100 bp, about 50 bp, and the like.

In certain embodiments, the non-coding target nucleic acid sequence may include any enhancer sequence within about a 5 kb region upstream of the transcriptional start site of the target gene, including about 4.5 kb, about 4 kb, about 3.5 kb, about 3 kb, about 2.5 kb, about 2 kb, about 1.5 lcb, and the like. In other embodiments, the non-coding target nucleic acid sequence may include the first intron sequence downstream of the transcriptional start site of the target gene.

The strands of the saRNA agent may have terminal (5′ or 3′) overhang regions of any length that are non-base pairing nucleotide resulting from one strand extending beyond the other strand within a doubled stranded polynucleotide. In addition, the overhang regions are also not complementary (a region of non-complementarity) to the non-coding sequence of the gene. If they have overhang regions, these regions in representative embodiments are 8 nucleotides or fewer in length, 7 nucleotides or fewer in length, 6 nucleotides or fewer in length, including about 5 nucleotides, about 4 nucleotides, such as about 3 nucleotides or fewer in length, including about two nucleotides in length, and about one nucleotide in length. In such embodiments, the regions are further described by the following formula:

3′-N(1+n)-saRNA-5′, or

5′-N(1+n)-saRNA-3′ wherein N is any nucleotide, including naturally-occurring or non-naturally-occurring, genetically encodable or non-genetically encodable, residue and n is any integer from 0 to 7.

In representative embodiments, a modulation (e.g., increase) in gene expression results in at least about a 2-fold increase or more in transcription associated with a nucleic acid sequence, as compared to a control, e.g., in the absence of the saRNA agent. In some embodiments, the increase in gene expression results in at least about a 2.5-fold increase or more, at least about a 3-fold increase or more, at least about a 3.5-fold increase or more, at least about a 4-fold increase or more, at least about a 4.5-fold increase or more, at least about a 5-fold increase or more, at least about a 5.5-fold increase or more, at least about a 6-fold increase or more, at least about a 6.5-fold increase or more, at least about a 7-fold increase or more, at least about a 7.5-fold increase or more at least about a 8-fold increase or more, and up to about 10-fold increase or more, including about 15-fold increase or more, about 20-fold increase or more, such as 25-fold increase or more. An increase in gene expression or activity can be measured by any of a variety of methods well known in the art. Suitable methods of examining gene expression or activity include measuring nucleic acid transcription level, mRNA level, enzymatic activity, methylation state, chromatin state or configuration, or other measure of nucleic acid activity or state in a cell or biological system. In some embodiments, the gene expression is decreased.

After introduction of a saRNA agent into a cell, a decrease in histone methylation (e.g., at a lysine) occurs. Accordingly, introduction of the saRNA agent in the cell results in demethylation of a histone molecule, such as histone 3, usually at the lysine residue, e.g., a lysine 9 residue.

In one aspect, the invention features a method of selectively modulating (e.g., increasing or decreasing, preferably increasing) synthesis of a target transcript of a gene in a mammalian cell, wherein the target transcript is predetermined to be in need of increased synthesis, the method comprising the steps of: contacting the cell with a polynucleotide oligomer of 12-28 bases complementary to a region within a target promoter of the gene under conditions whereby the oligomer selectively increases synthesis of the target transcript; and detecting resultant selective increased synthesis of the target gene.

In one embodiment, the region is located between nucleotides −100 to +25 relative to a transcription start site of the gene. In further embodiments, the region is located between nucleotides −50 to +25, −30 to +17, and −15 to +10, relative to a transcription start site of the gene. In a particular embodiment, the region includes nucleotides −9 to +2 relative to a transcription start site of the gene. In a particular embodiment, the region includes a transcription start site of the gene.

In one embodiment, the target promoter is the promoter of the target transcript. In another embodiment, the target promoter is the promoter of an isoform of the target transcript. In further embodiments, the target promoter is both the promoter of the target transcript and the promoter of an isoform of the target transcript.

In one embodiment, the region within the promoter of the gene is selected from a partially single-stranded structure, a non-B-DNA structure, an AT-rich sequence, a cruciform loop, a G-quadruplex, a nuclease hypersensitive elements (NHE), and a region located between nucleotides −100 to +25 relative to a transcription start site of the gene.

Preferred AT-rich sequences are found in stretches of DNA where local melting occurs, such as the promoters of genes where protein machinery must gain access to single-stranded regions, and preferably comprise the TATA box of the gene, and/or at least 60% or 70% A+T.

Preferred cruciform structures are formed from palindromic genomic sequences forming a hairpin structure on each strand, wherein the repeated sequences are separated by a stretch of non-palindromic DNA providing a single-stranded loop at the end of each of the hairpins of the cruciform.

Preferred G-quadruplex structures are identified in promoter regions of mammalian genes and are implicated in transcription regulation. For example the nuclease hypersensitivity element III of the c-MYC oncogene promoter is involved in controlling transcription and comprises a pyrimidine-rich and purine-rich sequences on the coding and noncoding strands, respectively, that can adopt I-motif and G-quadruplex structures, respectively. Stabilization of the G-quadruplex has been shown to lead to repression of c-MYC (see e.g. Siddiqui-Jain, 2002).

In one embodiment, the region targeted is located between nucleotides −100 to +25 relative to a transcription start site of the gene. In certain preferred embodiments of the method, the region is located on the template strand between nucleotides −30 to +17 relative to a transcription start site of the gene. In another embodiment, the region is located between nucleotides −15 to +10 relative to a transcription start site of the gene. In a further embodiment, the region includes nucleotides −9 to +2 relative to a transcription start site of the gene. In certain preferred embodiments, the region includes a transcription start site of the gene. In other embodiments, the region does not include any sequence downstream from the transcription start, e.g. the sequence is located between nucleotides −100 to +1. The oligomers used in the subject invention target genomic sequence and not mRNA.

In certain embodiments, the gene is known to encode and/or express one or more isoforms of the target transcript, and the method of the invention selectively increases synthesis of the target transcript over basal expression levels and/or control condition levels, while synthesis of the isoform(s) of the target transcript may decrease, increase, or stay the same. The target transcript and the isoform(s) may share the same promoter and/or transcription start site, or they may have different promoters and/or transcription start sites. Accordingly, in various embodiments, the target promoter is (1) the promoter of the target transcript, (2) the promoter of an isoform of the target transcript, or (3) is both the promoter of the target transcript and the promoter of an isoform of the target transcript. Numerous genes are known to express multiple isoforms; examples include p53 (Bourdon, 2005), PTEN (Sharrard and Maitland, 2000), Bcl-2-related genes (Akgul, 2004), and survivin (Caldas et al, 2005). For example, the methods can be used to increase expression of one target transcript by directing oligomers to the transcription start site of an isoform. Where synthesis of the target transcript is increased, and synthesis of the isoform is inhibited, the method effectively and selectively modulates relative isoform synthesis in the host cell. Hence, increased synthesis of predetermined desirous or underexpressed isoforms can be coupled with decreased synthesis of predetermined undesirable or overexpressed isoforms. As exemplified with p53[beta]/p53 below, this embodiment can be used to effect a predetermined isoform switch in the host cells.

In some embodiments, the oligonucleotide agent (e.g., saRNA) is modified, for example, to further stabilize against nucleolytic degradation. Exemplary modifications include a nucleotide base or modification of a sugar moiety. The oligonucleotide agent can include modified linker agent such as a phosphorothioate in at least the first, second, or third internucleotide linkage at the 5′ or 3′ end of the nucleotide sequence. In one embodiment, the oligonucleotide agent includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In a particularly preferred embodiment, the oligonucleotide agent includes at least one 2′-O-methyl-modified nucleotide, and in some embodiments, all of the nucleotides of the oligonucleotide agent include a 2′-O-methyl modification. In some embodiments, the sugar moiety of the nucleotide can be replaced, for example, with a non-sugar moiety such as a PNA.

The oligonucleotide agent can be further modified so as to be attached to a ligand. The ligand can be selected, for example, to improve stability, distribution or cellular uptake of the agent, e.g., cholesterol or folate.

The oligonucleotide agent can further be in isolated form or can be part of a pharmaceutical composition used for the methods described herein, particularly as a pharmaceutical composition formulated for parental administration. The pharmaceutical compositions can contain one or more oligonucleotide agents, and in some embodiments, will contain two or more oligonucleotide agents, each one directed to a different miRNA.

In one aspect, the invention features a method of modulating (e.g., increasing or decreasing, preferably increasing) the transcription of a gene in a cell of a subject, e.g., a human subject. The method includes the step of administering a oligonucleotide agent described herein (e.g., an saRNA such as a modified saRNA) to the subject, where the oligonucleotide agent is substantially single-stranded and includes a sequence that is substantially complementary to 12 to 23 contiguous nucleotides, and preferably 15 to 23 contiguous nucleotides, of a target sequence.

In another aspect, the invention features a method of making a oligonucleotide agent described herein, such as a modified single-stranded oligonucleotide agent, described herein. In one embodiment, the method includes synthesizing an oligonucleotide agent, including incorporating a nucleotide modification that stabilizes the oligonucleotide agent (e.g., saRNA) against nucleolytic degradation.

In another aspect, the invention features a pharmaceutical composition including an oligonucleotide agent (e.g., saRNA), such as a single-stranded oligonucleotide agent, described herein, and a pharmaceutically acceptable carrier. In a preferred embodiment, the saRNA, such as a single-stranded oligonucleotide agent, included in the pharmaceutical composition hybridizes to a non-coding nucleic acid sequence of a gene, for example, a promoter sequence.

Preferably, an oligonucleotide agent (e.g., saRNA) will include a ligand that is selected to improve stability, distribution or cellular uptake of the agent. Compositions featured in the invention can include conjugated single-stranded oligonucleotide agents as well as conjugated monomers that are the components of or can be used to make the conjugated oligonucleotide agents. The conjugated oligonucleotide agents can modify gene expression by targeting and binding to a nucleic acid. Exemplary ligands include Cholesterol and lipophilic conjugates (e.g., lithocholic-oleyl, lauroyl, docosnyl, stearoyl, palmitoyl, myristoyl, oleoyl, linoleoyl), galactose and Gal-NAc conjugates and clusters, folate and transferring.

In a preferred embodiment, the ligand is a lipophilic moiety, e.g., cholesterol, lithocholic-oleyl, lauroyl, docosnyl, stearoyl, palmitoyl, myristoyl, oleoyl, or linoleoyl, which enhances entry of the oligonucleotide agent (e.g., saRNA), such as a single-stranded oligonucleotide agent, into a cell, such as a hepatocyte, synoviocyte, myocyte, keratinocyte, leukocyte, endothelial cell (e.g., a kidney cell), B-cell, T-cell, epithelial cell, mesodermal cell, myeloid cell, neural cell, neoplastic cell, mast cell, or fibroblast cell. In some embodiments, a myocyte is a smooth muscle cell or a cardiac myocyte. A fibroblast cell can be a dermal fibroblast, and a leukocyte can be a monocyte. In another embodiment, the cell is from an adherent tumor cell line derived from a tissue, such as bladder, lung, breast, cervix, colon, pancreas, prostate, kidney, liver, skin, or nervous system (e.g., central nervous system). In some preferred embodiments, the ligand is a folate ligand.

In one embodiment, the oligonucleotide agent (e.g., an saRNA), such as a single-stranded oligonucleotide agent to which a lipophilic moiety is conjugated is used to increase expression of a gene in a cell that is not part of a whole organism, such as when the cell is part of a primary cell line, secondary cell line, tumor cell line, or transformed or immortalized cell line. Cells that are not part of a whole organism can be used in an initial screen to determine if an saRNA, such as a single-stranded oligonucleotide agent, is effective in increasing target gene expression levels. A test in cells that are not part of a whole organism can be followed by test of the saRNA in a whole animal. In some embodiments, the saRNA that is conjugated to a lipophilic moiety is administered to an organism, or contacted with a cell that is not part of an organism, in the absence of (or in a reduced amount of) other reagents that facilitate or enhance delivery, e.g., a compound which enhances transit through the cell membrane. (A reduced amount can be an amount of such reagent which is reduced in comparison to what would be needed to get an equal amount of nonconjugated agent into the target cell). For example, the saRNA that is conjugated to a lipophilic moiety is administered to an organism, or contacted with a cell that is not part of an organism, in the absence (or reduced amount) of (i) an additional lipophilic moiety; (ii) a transfection agent (e.g., an ion or other substance which substantially alters cell permeability to an oligonucleotide agent); or (iii) a commercial transfecting agent such as Lipofectamine™ (Invitrogen, Carlsbad, Calif.), Lipofectamine 2000™, TransIT-TKO™ (Mirus, Madison, Wis.), FuGENE 6 (Roche, Indianapolis, Ind.), polyethylenimine, X-tremeGENE Q2 (Roche, Indianapolis, Ind.), DOTAP, DOSPER, Metafectene™ (Biontex, Munich, Germany), and the like.

Exemplary delivery vehicles for an oligonucleotide agent featured herein, include lipid (e.g., cationic lipid) containing vehicles (e.g., liposomes), viral containing vehicles (e.g., vectors), polymer containing vehicles (e.g., biodegradable polymers or dendrimers), and peptide containing vehicles (e.g., a penetration peptide), exosomes, and bacterially-derived, intact minicells. In a preferred example the delivery vehicle includes more than one component. For example, it can include one or more lipid moieties, one or more peptides, one or more polymers, one or more viral vectors, or a combination thereof.

In a preferred embodiment, the delivery vehicle is an association complex such as a liposome. A liposome generally includes a plurality of components such as one or more of a cationic lipid (e.g., an amino lipid), a targeting moiety, a fusogenic lipid, a PEGylated lipid. In some embodiments, the PEG-lipid is a targeted PEG-lipid. For example, a liposome can include a nucleic acid and an amine-lipid and a PEGylated lipid. In some embodiments, the PEG-lipid is a targeted PEG-lipid. In some embodiments, the PEG lipid has the following structure:

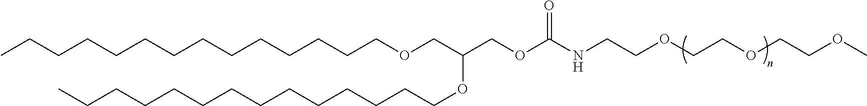

wherein the repeating PEG moiety has an average molecular weight of 2000 with n value between 42 and 47.

In some embodiments, the preparation also includes a structural moiety such as cholesterol.

Exemplary Candidate Delivery Vehicles

An oligonucleotide agent can be delivered using a variety of delivery vehicles including those containing one or more of the following: cationic lipids, cationic liposomes, neutral and zwitterionic lipids and liposomes, peptides (neutral, anionic and cationic; hydrophobic), dendrimers (neutral, anionic and cationic), polymers, emulsions, intralipids, omega-3 and related natural formulations, microemulsions and nanoemulsions, nanoparticles, nanosystems with targeting groups, nanosystems with endosomal releasing groups, polymeric micelles, polymeric vesicles, PEIs and polyamines, lipophilic polyamines, and hydrogels.

Exemplary delivery vehicles include peptide containing vehicles, collagen containing vehicles, viral vector containing vehicles, polymer containing vehicles, lipid containing (e.g., cationic lipids, PEG containing lipids, etc.). In some embodiments, the delivery vehicle includes a combination of one or more of the delivery components described above. Exemplary delivery vehicles, which can be evaluated using a screening model described herein include, but are not limited to the following: Exosomes such as those described in US 20070298118; bacterially-derived, intact minicells, for example, as described in US 20070298056; complexes including RNA and peptides such as those described in US 20070293657; cationic lipids, non-cationic lipids, and lipophilic delivery-enhancing compounds such as those described in US 20070293449); Carbohydrate-Derivatized Liposomes (e.g., as described in US 20070292494); siRNA-hydrophilic polymer conjugates (e.g., as described in US 20070287681); lipid and polypeptide based systems such as those described in US 20070281900; organic cation containing systems such as those described in US 20070276134 and US 20070213257; cationic peptide containing systems such as those described in US 20070275923; polypeptide containing systems such as those disclosed in US 20060040882; virus-phage particle containing systems such as those described in US 20070274908; elastin-like polymer containing systems such as those described in US 20070265197; non-immunogenic, hydrophilic/cationic block copolymers such as those described in US 20070259828; carrier linked conjugate containing systems such as those described in US 20070258993; biodegradable cationic polymer containing systems such as those described in US 20070243157; chemically modified polycation polymer containing systems such as those described in US 20070231392; collagen containing systems such as those described in US 20070218038; glycopolymer-based particle containing systems such as those described in US 20070202076; biologically active block copolymer containing systems such as those described in US 20070155907; nanoparticle containing systems such as those described in US 20070155658; amphoteric liposome containing systems such as those described in US 20070104775; lipid carrier containing systems such as those described in US 20070087045; electroporation systems such as those described in US 20070059832; macromer-melt formulations such as those described in US 20070053954; liposome containing systems such as those described in US 20070042031 and US 20050002999 and US 20050002998; lipid based formulations such as those described in US 20060008910; US 20050014962; US 20060240093, US 20050064595, and US 20060083780; polymer conjugate containing systems such as those described in US 20070041932 and US 20050008617; hydrophobic nanotube and nanoparticle containing systems such as those described in US 20060275371; functional synthetic molecule and macromolecule containing systems such as those described in US 20060241071; polymeric micelle containing systems such as those disclosed in US 20060240092; sugar-modified liposome containing systems such as those described in US 20060193906; cyclic amidinium-containing systems such as those described in US 20060039860 and US 20030220289; peptide containing compositions such as those described in US 20060035815 and US 20050239687; viral vector containing systems such as those described in US 20060009408, US 20030157691, and tRNA vector systems such as those described in US 20050203047; nanocell drug delivery systems such as those described in US 20050266067; polymerized formamide containing systems such as those described in US 20050265957; intranasal delivery systems such as those described in US 20050265927; nanoparticle systems such as those described in US 20050260276; biodegradable polymer-peptide containing systems such as those described in US 20050191746 and biodegradable polyacetal containing systems such as those described in US 20050080033; biodegradable cationic polymer containing systems such as those described in US 20060258751; biodegradable poly(beta-amino ester) containing systems such as those described in US 20040071654; polyethyleneglycol-modified lipid containing systems such as those described in US 20050175682; virally-encoded RNA systems such as those described in US 20050171041, US 20040023390, US 20030138407; adenoviral vector systems such as those described in US 20040161848 and US 20040096843; carrier complex containing systems such as those described in US 20050158373; compositions comprising amphipathic compounds and polycations such as those described in US 20050143332, US 20040137064, and US 20030125281; delivery peptide and dendrimer containing compositions such as those described in US 20040204377; polyampholyte containing compositions such as those described in US 20040162235; and microcapsule containing systems such as those described in US 20040115254 and formulations described in PCT/US2007/080331. Each of the references above is incorporated by reference herein in its entirety.

In some preferred embodiments one or more of the delivery vehicles can be formed into a particle such as a liposome or other association complex. The nucleic acid-based agent can be encapsulated or partially encapsulated in the particle delivery vehicle. In some embodiments, the nucleic acid-based agent is admixed with one or more delivery vehicles described herein. In some preferred embodiments, the cationic lipid is a polyamine cationic lipid. In some preferred embodiments, the delivery vehicle includes one or more of TETA-5LAP, DLinDMA, and Analogs with PEGS and Cholesterol.

In some embodiments, the nucleic acid-based agent is bound to a delivery vehicle described herein. For example, the nucleic acid-based agent can be bound to a delivery vehicle through hydrostatic interactions, ionic interactions, hydrogen bonding interactions or through a covalent bond.

In some embodiments, the nucleic acid-based agent is entrapped or entrained within a delivery vehicle.

Association Complexes

Association complexes can be used to administer a nucleic acid based therapy such as an oligonucleotide agent described herein. The association complexes disclosed herein can be useful for packaging an oligonucleotide.

Association complexes can include a plurality of components. In some embodiments, an association complex such as a liposome can include an oligonucleotide agent described herein, a cationic lipid such as an amino lipid. In some embodiments, the association complex can include a plurality of therapeutic agents, for example two or three single or double stranded nucleic acid moieties targeting more than one gene or different regions of the same gene. Other components can also be included in an association complex, including a PEG-lipid or a structural component, such as cholesterol. In some embodiments the association complex also includes a fusogenic lipid or component and/or a targeting molecule. In some preferred embodiments, the association complex is a liposome including an oligonucleotide agent such as an saRNA, a lipid, a PEG-lipid, and a structural component such as cholesterol. In some embodiments, the PEG lipid has the following structure:

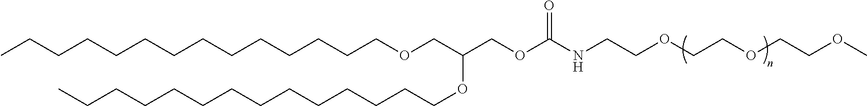

wherein the repeating PEG moiety has an average molecular weight of 2000 with n value between 42 and 47.

In a preferred embodiment, the saRNA is suitable for delivery to a cell in vivo, e.g., to a cell in an organism. In another embodiment, the saRNA is suitable for delivery to a cell in vitro, e.g., to a cell in a cell line.

An saRNA to which a lipophilic moiety is attached can have a sequence complementary to a non-coding nucleic acid sequence of a target (e.g., a promoter region) and can be delivered to any cell type described herein, e.g., a cell type in an organism, tissue, or cell line. Delivery of the saRNA can be in vivo, e.g., to a cell in an organism, or in vitro, e.g., to a cell in a cell line.

In another aspect, the invention provides compositions including single-stranded oligonucleotide agents described herein, and in particular, compositions including an saRNA to which a lipophilic moiety is conjugated, e.g., a lipophilic conjugated saRNA that hybridizes to a non-coding nucleic acid sequence of a target (e.g., a target gene). In a preferred embodiment the composition is a pharmaceutically acceptable composition.

In one embodiment the composition is suitable for delivery to a cell in vivo, e.g., to a cell in an organism. In another aspect, the saRNA is suitable for delivery to a cell in vitro, e.g., to a cell in a cell line.

“Substantially complementary” means that two sequences are substantially complementary that a duplex can be formed between them. The duplex may have one or more mismatches but the region of duplex formation is sufficient to down-regulate expression of the target nucleic acid. The region of substantial complementarity can be perfectly paired. In other embodiments, there will be nucleotide mismatches in the region of substantial complementarity. In a preferred embodiment, the region of substantial complementarity will have no more than 1, 2, 3, 4, or 5 mismatches.

The oligonucleotide agents featured in the invention include oligomers or polymers of ribonucleic acid (RNA) or deoxyribonucleic acid (DNA) or both or modifications thereof. This term includes oligonucleotides composed of naturally occurring nucleobases, sugars, and covalent internucleoside (backbone) linkages as well as oligonucleotides having non-naturally-occurring portions that function similarly. Such modified or substituted oligonucleotides are often preferred over native forms because of desirable properties such as, for example, enhanced cellular uptake, enhanced affinity for nucleic acid target, and/or increased stability in the presence of nucleases. The oligonucleotide agents can be about 12 to about 30 nucleotides long, e.g., about 15 to about 25, or about 18 to about 25 nucleotides long (e.g., about 19, 20, 21, 22, 23, 24 nucleotides long).

The oligonucleotide agents described herein, e.g., saRNAs, featured in the invention can target a non-coding sequence of a target gene (e.g., a promoter), in a subject. Such single-stranded oligonucleotide agents can be useful for the treatment of diseases involving biological processes that are regulated by gene transcription, including developmental timing, differentiation, apoptosis, cell proliferation, organ development, and metabolism.

For increased nuclease resistance and/or binding affinity to the target, the oligonucleotide agents described herein can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to the target. The inclusion of pyranose sugars in the oligonucleotide backbone can also decrease endonucleolytic cleavage. An oligonucleotide agent featured in the invention, e.g., a supermir, can be further modified by including a 3′ cationic group, or by inverting the nucleoside at the 3′-terminus with a 3′-3′ linkage. In another alternative, the 3′-terminus can be blocked with an aminoalkyl group, e.g., a 3′ C5-aminoalkyl dT. Other 3′ conjugates can inhibit 3′-5′ exonucleolytic cleavage. While not being bound by theory, a 3′ conjugate, such as naproxen or ibuprofen, may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

The 5′-terminus can be blocked with an aminoalkyl group, e.g., a 5′-O-alkylamino substituent. Other 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′ end of the oligonucleotide. Even small alkyl chains, aryl groups, or heterocyclic conjugates or modified sugars (D-ribose, deoxyribose, glucose etc.) can block 3′-5′-exonucleases.

In one embodiment, an oligonucleotide agent, such as an saRNA, includes a modification that improves targeting, e.g. a targeting modification described herein. Examples of modifications that target single-stranded oligonucleotide agents to particular cell types include carbohydrate sugars such as galactose, N-acetylgalactosamine, mannose; vitamins such as folates; other ligands such as RGDs and RGD mimics; and small molecules including naproxen, ibuprofen or other known protein-binding molecules.

An oligonucleotide agent, such as an saRNA, featured in the invention can be constructed using chemical synthesis and/or enzymatic ligation reactions using procedures known in the art. For example, an oligonucleotide agent can be chemically synthesized using naturally occurring nucleotides or variously modified nucleotides designed to increase the biological stability of the molecules or to increase the physical stability of the duplex formed between the oligonucleotide agent and target nucleic acids, e.g., phosphorothioate derivatives and acridine substituted nucleotides can be used. Other appropriate nucleic acid modifications are described herein. Alternatively, the oligonucleotide agent can be produced biologically using an expression vector into which a nucleic acid has been subcloned in an antisense orientation (i.e., RNA transcribed from the inserted nucleic acid will be of an antisense orientation to a target nucleic acid of interest).

Chemical Definitions

The term “halo” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. The term “haloalkyl” refers to an alkyl in which one or more hydrogen atoms are replaced by halo, and includes alkyl moieties in which all hydrogens have been replaced by halo (e.g., perfluoroalkyl). Alkyl and haloalkyl groups may be optionally inserted with O, N, or S. The terms “aralkyl” refers to an alkyl moiety in which an alkyl hydrogen atom is replaced by an aryl group. Aralkyl includes groups in which more than one hydrogen atom has been replaced by an aryl group. Examples of “aralkyl” include benzyl, 9-fluorenyl, benzhydryl, and trityl groups.

The term “alkenyl” refers to a straight or branched hydrocarbon chain containing 2-8 carbon atoms and characterized in having one or more double bonds. Examples of a typical alkenyl include, but not limited to, allyl, propenyl, 2-butenyl, 3-hexenyl and 3-octenyl groups. The term “alkynyl” refers to a straight or branched hydrocarbon chain containing 2-8 carbon atoms and characterized in having one or more triple bonds. Some examples of a typical alkynyl are ethynyl, 2-propynyl, and 3-methylbutynyl, and propargyl. The sp² and sp³ carbons may optionally serve as the point of attachment of the alkenyl and alkynyl groups, respectively.

The terms “alkylamino” and “dialkylamino” refer to —NH(alkyl) and —NH(alkyl)₂ radicals respectively. The term “aralkylamino” refers to a —NH(aralkyl) radical. The term “alkoxy” refers to an —O-alkyl radical, and the terms “cycloalkoxy” and “aralkoxy” refer to an —O-cycloalkyl and O-aralkyl radicals respectively. The term “siloxy” refers to a R₃SiO— radical. The term “mercapto” refers to an SH radical. The term “thioalkoxy” refers to an —S—alkyl radical.

The term “alkylene” refers to a divalent alkyl (i.e., —R—), e.g., —CH₂—, —CH₂CH₂—, and —CH₂CH₂CH₂—. The term “alkylenedioxo” refers to a divalent species of the structure —O—R—O—, in which R represents an alkylene.

The term “aryl” refers to an aromatic monocyclic, bicyclic, or tricyclic hydrocarbon ring system, wherein any ring atom can be substituted. Examples of aryl moieties include, but are not limited to, phenyl, naphthyl, anthracenyl, and pyrenyl.

The term “cycloalkyl” as employed herein includes saturated cyclic, bicyclic, tricyclic, or polycyclic hydrocarbon groups having 3 to 12 carbons, wherein any ring atom can be substituted. The cycloalkyl groups herein described may also contain fused rings. Fused rings are rings that share a common carbon-carbon bond or a common carbon atom (e.g., spiro-fused rings). Examples of cycloalkyl moieties include, but are not limited to, cyclohexyl, adamantyl, and norbornyl, and decalin.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom can be substituted. The heterocyclyl groups herein described may also contain fused rings. Fused rings are rings that share a common carbon-carbon bond or a common carbon atom (e.g., spiro-fused rings). Examples of heterocyclyl include, but are not limited to tetrahydrofuranyl, tetrahydropyranyl, piperidinyl, morpholino, pyrrolinyl and pyrrolidinyl.

The term “cycloalkenyl” as employed herein includes partially unsaturated, nonaromatic, cyclic, bicyclic, tricyclic, or polycyclic hydrocarbon groups having 5 to 12 carbons, preferably 5 to 8 carbons, wherein any ring atom can be substituted. The cycloalkenyl groups herein described may also contain fused rings. Fused rings are rings that share a common carbon-carbon bond or a common carbon atom (e.g., spiro-fused rings). Examples of cycloalkenyl moieties include, but are not limited to cyclohexenyl, cyclohexadienyl, or norbornenyl.

The term “heterocycloalkenyl” refers to a partially saturated, nonaromatic 5-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom can be substituted. The heterocycloalkenyl groups herein described may also contain fused rings. Fused rings are rings that share a common carbon-carbon bond or a common carbon atom (e.g., spiro-fused rings). Examples of heterocycloalkenyl include but are not limited to tetrahydropyridyl and dihydropyran.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom can be substituted. The heteroaryl groups herein described may also contain fused rings that share a common carbon-carbon bond.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Suitable substituents include, without limitation, alkyl, alkenyl, alkynyl, alkoxy, halo, hydroxy, cyano, nitro, amino, SO₃H, sulfate, phosphate, perfluoroalkyl, perfluoroalkoxy, methylenedioxy, ethylenedioxy, carboxyl, oxo, thioxo, imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, and combinations thereof), ester (alkyl, aralkyl, heteroaralkyl), amide (mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl. In one aspect, the substituents on a group are independently any one single, or any subset of the aforementioned substituents.

The terms “adeninyl, cytosinyl, guaninyl, thyminyl, and uracilyl” and the like refer to radicals of adenine, cytosine, guanine, thymine, and uracil.

A “protected” moiety refers to a reactive functional group, e.g., a hydroxyl group or an amino group, or a class of molecules, e.g., sugars, having one or more functional groups, in which the reactivity of the functional group is temporarily blocked by the presence of an attached protecting group. Protecting groups useful for the monomers and methods described herein can be found, e.g., in Greene, T. W., Protective Groups in Organic Synthesis (John Wiley and Sons: New York), 1981, which is hereby incorporated by reference.

saRNA Structure

An saRNA, such as a single-stranded oligonucleotide agent, featured in the invention includes a region sufficient complementarity to the non-coding region of a target gene (e.g., a promoter sequence), and is of sufficient length in terms of nucleotides, such that the saRNA forms a duplex with the target nucleic acid. The supermir can modulate the function of the targeted sequence. For example, oligonucleotide agent can increase gene expression of the target gene.

For ease of exposition the term nucleotide or ribonucleotide is sometimes used herein in reference to one or more monomeric subunits of an oligonucleotide agent. It will be understood herein that the usage of the term “ribonucleotide” or “nucleotide” herein can, in the case of a modified RNA or nucleotide surrogate, also refer to a modified nucleotide, or surrogate replacement moiety at one or more positions.

An saRNA featured in the invention is, or includes, a region that is at least partially, and in some embodiments fully, complementary to the target sequence (e.g., a promoter region of a target gene). It is not necessary that there be perfect complementarity between the saRNA and the target, but the correspondence must be sufficient to enable the oligonucleotide agent to modulate (e.g., increase) target gene expression.

An saRNA agent will preferably have one or more of the following properties:
(1) it will have a 5′ modification that includes one or more phosphate groups or one or more analogs of a phosphate group;
(2) it will, despite modifications, even to a very large number of bases specifically base pair and form a duplex structure with a homologous target sequence of sufficient thermodynamic stability to allow modulation of the activity of the targeted gene;
(3) it will, despite modifications, even to a very large number, or all of the nucleosides, still have “RNA-like” properties, i.e., it will possess the overall structural, chemical and physical properties of an RNA molecule, even though not exclusively, or even partly, of ribonucleotide-based content. For example, all of the nucleotide sugars can contain e.g., 2′OMe, 2′ fluoro in place of 2′ hydroxyl. This deoxyribonucleotide-containing agent can still be expected to exhibit RNA-like properties. While not wishing to be bound by theory, an electronegative fluorine prefers an axial orientation when attached to the C2′ position of ribose. This spatial preference of fluorine can, in turn, force the sugars to adopt a C_{3′}-endo pucker. This is the same puckering mode as observed in RNA molecules and gives rise to the RNA-characteristic A-family-type helix. Further, since fluorine is a good hydrogen bond acceptor, it can participate in the same hydrogen bonding interactions with water molecules that are known to stabilize RNA structures. (Generally, it is preferred that a modified moiety at the 2′ sugar position will be able to enter into hydrogen-bonding which is more characteristic of the 2′-OH moiety of a ribonucleotide than the 2′-H moiety of a deoxyribonucleotide. A preferred supermir will: exhibit a C_{3′}-endo pucker in all, or at least 50, 75, 80, 85, 90, or 95% of its sugars; exhibit a C_{3′}-endo pucker in a sufficient amount of its sugars that it can give rise to a the RNA-characteristic A-family-type helix; will have no more than 20, 10, 5, 4, 3, 2, or 1 sugar which is not a C_{3′}-endo pucker structure.

Preferred 2′-modifications with C3′-endo sugar pucker include:

2′-OH, 2′-O-Me, 2′-O-methoxyethyl, 2′-O-aminopropyl, 2′-F, 2′-O—CH2-CO—NHMe, 2′-O—CH2-CH2-O—CH2-CH2-N(Me)2, and LNA

Preferred 2′-modifications with a C2′-endo sugar pucker include:

2′-H, 2′-Me, 2′-S-Me, 2′-Ethynyl, 2′-ara-F.

Sugar modifications can also include L-sugars and 2′-5′-linked sugars.

As used herein, “specifically hybridizable” and “complementary” are terms that are used to indicate a sufficient degree of complementarity such that stable and specific binding occurs between a supermir of the invention and a target RNA molecule, e.g., an mRNA target of an endogenous miRNA or a pre-miRNA. Specific binding requires a sufficient lack of complementarity to non-target sequences under conditions in which specific binding is desired, i.e., under physiological conditions in the case of in vivo assays or therapeutic treatment, or in the case of in vitro assays, under conditions in which the assays are performed. It has been shown that a single mismatch between targeted and non-targeted sequences are sufficient to provide discrimination for siRNA targeting of an mRNA (Brummelkamp et al., Cancer Cell, 2002, 2:243).

In one embodiment, an oligonucleotide agent featured in the invention, e.g., an saRNA, is “sufficiently complementary” to a target sequence (e.g., a non-coding nucleic acid sequence of a gene such as a promoter region), such that the oligonucleotide agent increases transcription of the gene. In another embodiment, the oligonucleotide agent is “exactly complementary” (excluding the SRMS containing subunit(s)) to a target RNA, e.g., the target RNA and the oligonucleotide agent can anneal to form a hybrid made exclusively of Watson-Crick base pairs in the region of exact complementarity. A “sufficiently complementary” target RNA can include a region (e.g., of at least 7 nucleotides) that is exactly complementary to a target RNA. Moreover, in some embodiments, the oligonucleotide agent specifically discriminates a single-nucleotide difference. In this case, the oligonucleotide agent only down-regulates gene expression if exact complementarity is found in the region of the single-nucleotide difference. As used herein, the term “complementary,” without further modification, means sufficiently complementary.

Oligonucleotide agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, preferably different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone. Examples of all of the above are discussed herein.

In some embodiments, the oligonucleotide agent is modified with one of the following modifications: modification of the nucleotide backbone such as modification of the phosphate linker or replacement of the phosphate linker; modification of the sugar moiety such as modification of the 2′ hydroxyl on the ribose; replacement of the sugar moiety such as ribose or deoxyribose with a different chemical structure such as a PNA structure; or modification of the nucleobase for example modification to a universal base or G-clamp.

As nucleic acids are polymers of subunits or monomers, many of the modifications described below occur at a position which is repeated within a nucleic acid, e.g., a modification of a base, or a phosphate moiety, or a non-linking O of a phosphate moiety. In some cases the modification will occur at all of the subject positions in the nucleic acid but in many, and in fact in most cases it will not. By way of example, a modification may only occur at a 3′ or 5′ terminal position, in a terminal region, e.g., at a position on a terminal nucleotide, or in the last 2, 3, 4, 5, or 10 nucleotides of a strand. The ligand can be attached at the 3′ end, the 5′ end, or at an internal position, or at a combination of these positions. For example, the ligand can be at the 3′ end and the 5′ end; at the 3′ end and at one or more internal positions; at the 5′ end and at one or more internal positions; or at the 3′ end, the 5′ end, and at one or more internal positions. For example, a phosphorothioate modification at a non-linking O position may only occur at one or both termini, or may only occur in a terminal region, e.g., at a position on a terminal nucleotide or in the last 2, 3, 4, 5, or 10 nucleotides of the oligonucleotide. The 5′ end can be phosphorylated.

Exemplary modifications of nucleotides are provided below:

Modifications and nucleotide surrogates are discussed below.

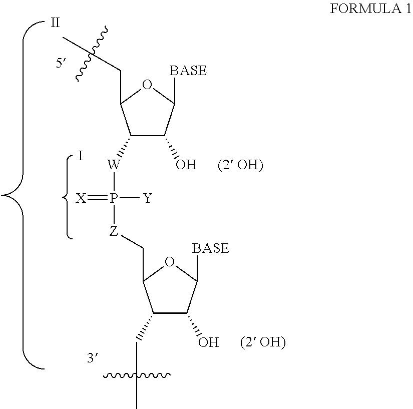

The scaffold presented above in Formula 1 represents a portion of a ribonucleic acid. The basic components are the ribose sugar, the base, the terminal phosphates, and phosphate internucleotide linkers. Where the bases are naturally occurring bases, e.g., adenine, uracil, guanine or cytosine, the sugars are the unmodified 2′ hydroxyl ribose sugar (as depicted) and W, X, Y, and Z are all O, Formula 1 represents a naturally occurring unmodified oligoribonucleotide.

Unmodified oligoribonucleotides may be less than optimal in some applications, e.g., unmodified oligoribonucleotides can be prone to degradation by e.g., cellular nucleases. Nucleases can hydrolyze nucleic acid phosphodiester bonds. However, chemical modifications to one or more of the above RNA components can confer improved properties, and, for example, can render oligoribonucleotides more stable to nucleases. Unmodified oligoribonucleotides may also be less than optimal in terms of offering tethering points for attaching ligands or other moieties to an oligonucleotide agent.

Modified nucleic acids and nucleotide surrogates can include one or more of:

(I) alteration, e.g., replacement, of one or both of the non-linking (X and Y) phosphate oxygens and/or of one or more of the linking (W and Z) phosphate oxygens (When the phosphate is in the terminal position, one of the positions W or Z will not link the phosphate to an additional element in a naturally occurring ribonucleic acid. However, for simplicity of terminology, except where otherwise noted, the W position at the 5′ end of a nucleic acid and the terminal Z position at the 3′ end of a nucleic acid, are within the term “linking phosphate oxygens” as used herein);

(II) alteration, e.g., replacement, of a constituent of the ribose sugar, e.g., of the 2′ hydroxyl on the ribose sugar, or wholesale replacement of the ribose sugar with a structure other than ribose, e.g., as described herein;

(III) wholesale replacement of the phosphate moiety (bracket I) with “dephospho” linkers;

(IV) modification or replacement of a naturally occurring base;

(V) replacement or modification of the ribose-phosphate backbone (bracket II);

(VI) ligands.

The terms replacement, modification, alteration, and the like, as used in this context, do not imply any process limitation, e.g., modification does not mean that one must start with a reference or naturally occurring ribonucleic acid and modify it to produce a modified ribonucleic acid but rather modified simply indicates a difference from a naturally occurring molecule.

It is understood that the actual electronic structure of some chemical entities cannot be adequately represented by only one canonical form (i.e. Lewis structure). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, the written and graphical descriptions of the embodiments of the present invention are made in terms of what the art recognizes as the predominant resonance form for a particular species. For example, any phosphoroamidate (replacement of a non-linking oxygen with nitrogen) would be represented by X═O and Y═N in the above figure.

Specific modifications are discussed in more detail below.

(I) The Phosphate Group

The phosphate group is a negatively charged species. The charge is distributed equally over the two non-linking oxygen atoms (i.e., X and Y in Formula 1 above). However, the phosphate group can be modified by replacing one of the oxygens with a different substituent. One result of this modification to RNA phosphate backbones can be increased resistance of the oligoribonucleotide to nucleolytic breakdown. Thus while not wishing to be bound by theory, it can be desirable in some embodiments to introduce alterations which result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. Unlike the situation where only one of X or Y is altered, the phosphorus center in the phosphorodithioates is achiral which precludes the formation of oligoribonucleotides diastereomers. Diastereomer formation can result in a preparation in which the individual diastereomers exhibit varying resistance to nucleases. Further, the hybridization affinity of RNA containing chiral phosphate groups can be lower relative to the corresponding unmodified RNA species. Thus, while not wishing to be bound by theory, modifications to both X and Y which eliminate the chiral center, e.g., phosphorodithioate formation, may be desirable in that they cannot produce diastereomer mixtures. Thus, X can be any one of S, Se, B, C, H, N, or OR (R is alkyl or aryl). Thus Y can be any one of S, Se, B, C, H, N, or OR (R is alkyl or aryl). Replacement of X and/or Y with sulfur is preferred. In some preferred embodiments, the phosphate is modified to a phosphorothioate, phosphorodithioate, boranophosphate, N3′-P5′ phosphoroamidate, thiophosphoroamidate, phosphoramidates, cationic phosphoramidate, phosphonoacetate, phosphonothioacetate, 3′-methylene phosphonate, or a methylphosphonate

The phosphate linker can also be modified by replacement of a linking oxygen (i.e., W or Z in Formula 1) with nitrogen (bridged phosphoroamidates), sulfur (bridged phosphorothioates) and carbon (bridged methylenephosphonates). The replacement can occur at a terminal oxygen (position W (3′) or position Z (5′)). Replacement of W with carbon or Z with nitrogen is preferred.

Exemplary modifications are also found in U.S. Ser. No. 11/170,798, which is incorporated herein by reference.

(II) The Sugar Group

A modified nucleotide agent can include modification of all or some of the sugar groups of the ribonucleic acid. For example, the 2′ hydroxyl group (OH) can be modified or replaced with a number of different “oxy” or “deoxy” substituents. While not being bound by theory, enhanced stability is expected since the hydroxyl can no longer be deprotonated to form a 2′ alkoxide ion. The 2′ alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge or ethylene bridge (e.g., 2′-4′-ethylene bridged nucleic acid (ENA)), to the 4′ carbon of the same ribose sugar; amino, O-AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e. deoxyribose sugars); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino), —NHC(O)R (R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality. Preferred substitutents are 2′-methoxyethyl, 2′-OCH3, 2′-O-allyl, 2′-C-allyl, and 2′-fluoro.

The sugar group can also contain one or more carbons that possess the opposite stereochemical configuration than that of the corresponding carbon in ribose. Thus, a modified RNA can include nucleotides containing e.g., arabinose, as the sugar.

Modified RNAs can also include “abasic” sugars, which lack a nucleobase at C-1′. These abasic sugars can also be further contain modifications at one or more of the constituent sugar atoms.

To maximize nuclease resistance, the 2′ modifications can be used in combination with one or more phosphate linker modifications (e.g., phosphorothioate). The so-called “chimeric” oligonucleotides are those that contain two or more different modifications.

The modification can also entail the wholesale replacement of a ribose structure with another entity (an SRMS) at one or more sites in the oligonucleotide agent.

In some preferred embodiments, the sugar modification includes one or more of 2′-OMe, 2′-F, 2′-O-MOE, 2′-O-thioMOE, 2′-O-AP, 2′-O-DMAOE, 2′-O-DMAEOE, 2′-O-NMA, 2′-O-DMAEA, 2′-O-GE, 2′-O-AE, 2′-O-DMAE, 2′-O-DMAP, 2′-O-ImBu, 2′-O-allyl, ANA, 2′-F-ANA, 3′-Modifications such as Terminal 3′-modifications, 2′-5′ linkages, 4′-Modifications, such as 4′-Thio sugar, 4′-F, 4′-C-aminoethyl, 4′-C-aminoalkyl, 5′-modifications, such as 5′-alkyl, O-alkyl, 5′-terminal modifications, such as 5′-hydroxymethyl, Bicyclic Sugars, LNA, ENA, α-L-LNA, and carbocyclic analogs of LNA.

In some preferred embodiments, the ribose is replaced with one or more of morpholino, a cationic Morpholino, a PNA, a PNA analog, HNA, or CeNA.

(III) Replacement of the Phosphate Group

The phosphate group can be replaced by non-phosphorus containing connectors (cf. Bracket I in Formula 1 above). While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. Preferred replacements include the methylenecarbonylamino and methylenemethylimino groups.

(IV) The Bases

Adenine, guanine, cytosine and uracil are the most common bases found in RNA. These bases can be modified or replaced to provide RNA's having improved properties. E.g., nuclease resistant oligoribonucleotides can be prepared with these bases or with synthetic and natural nucleobases (e.g., inosine, thymine, xanthine, hypoxanthine, nubularine, isoguanisine, or tubercidine) and any one of the above modifications. Alternatively, substituted or modified analogs of any of the above bases, e.g., “unusual bases” and “universal bases” described herein, can be employed. Examples include without limitation 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, and those disclosed by Englisch et al., Angewandte Chemie, International Edition, 1991, 30, 613.

In some preferred embodiments, the nucleotide agent includes one or more of the following base modifications: C-5 modified pyrimidine, N-2 modified purine, N-6 modified purine, C-8 modified purine, 2,6-Diaminopurine, a universal base, G-clamp, phenoxazines, or thiophenoxazine.

Exemplary base modifications are described in U.S. Ser. No. 11/186,915; U.S. Ser. No. 11/197,753; and U.S. Ser. No. 11/119,533, each of which is incorporated herein by reference.

(V) Replacement of Ribophosphate Backbone

Oligonucleotide-mimicking scaffolds can also be constructed wherein the phosphate linker and ribose sugar are replaced by nuclease resistant nucleoside or nucleotide surrogates (see Bracket II of Formula 1 above). While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone.

Examples include the mophilino, cyclobutyl, pyrrolidine and peptide nucleic acid (PNA) nucleoside surrogates. A preferred surrogate is a PNA surrogate.

(VI) Ligands

An oligonucleotide agent can be modified to include a ligand. For example, the modification ban be at 3′ or 5′ ends of an oligonucleotide, or internally. Such modifications can be at the 3′ end, 5′ end or both ends of the molecule. They can include modification or replacement of an entire terminal phosphate or of one or more of the atoms of the phosphate group. E.g., the 3′ and 5′ ends of an oligonucleotide can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a spacer. The atom of the spacer can connect to or replace the linking atom of the phosphate group or the C-3′ or C-5′ O, N, S or C group of the sugar. Alternatively, the spacer can connect to or replace the atom of a nucleotide surrogate (e.g., PNAs). These spacers or linkers can include e.g., —(CH₂)ₙ—, —(CH₂)ₙN—, —(CH₂)ₙO—, —(CH₂)ₙS—, O(CH₂CH₂O)ₙCH₂CH₂OH (e.g., n=3 or 6), abasic sugars, amide, carboxy, amine, oxyamine, oxyimine, thioether, disulfide, thiourea, sulfonamide, or morpholino, or biotin and fluorescein reagents. While not wishing to be bound by theory, it is believed that conjugation of certain moieties can improve transport, hybridization, and specificity properties. Again, while not wishing to be bound by theory, it may be desirable to introduce alterations (e.g., terminal alterations) that improve nuclease resistance. Other examples of terminal modifications include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic carriers (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles).

Modifications (e.g., terminal modifications) can be added for a number of reasons, including as discussed elsewhere herein to modulate activity or to modulate resistance to degradation. Preferred modifications include the addition of a methylphosphonate at the 3′-most terminal linkage; a 3′ C5-aminoalkyl-dT; 3′ cationic group; or another 3′ conjugate to inhibit 3′-5′ exonucleolytic degradation.

Modifications (e.g., terminal modifications) useful for modulating activity include modification of the 5′ end with phosphate or phosphate analogs. E.g., in preferred embodiments oligonucleotide agents are 5′ phosphorylated or include a phosphoryl analog at the 5′ terminus. 5′-phosphate modifications include those which are compatible with RISC mediated gene silencing. Suitable modifications include: 5′-monophosphate ((HO)2(O)P—O-5′); 5′-diphosphate ((HO)2(O)P—O—P(HO)(O)—O-5′); 5′-triphosphate ((HO)2(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)2(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, (OH)2(O)P-5′-CH2-), 5′-alkyletherphosphonates (R=alkylether=methoxymethyl (MeOCH2-), ethoxymethyl, etc., e.g. RP(OH)(O)—O-5′-).

Terminal modifications can also be useful for monitoring distribution, and in such cases the preferred groups to be added include fluorophores, e.g., fluorscein or an Alexa dye, e.g., Alexa 488. Terminal modifications can also be useful for enhancing uptake, useful modifications for this include cholesterol. Terminal modifications can also be useful for cross-linking anantagomir to another moiety; modifications useful for this include mitomycin C.

Exemplary lipophilic modifications include a cholesterol; a bile acid; and a fatty acid (e.g., lithocholic-oleyl, lauroyl, docosnyl, stearoyl, palmitoyl, myristoyl, oleoyl, linoleoyl). Other exemplary terminal modifications include the following, sugars, carbohydrates, folates and analogs thereof, PEGs, pluronics, PEI, endosomal releasing agents, cell surface targeting small molecules, cell surface targeting peptides (e.g. RGD and other phage display derived peptides), cell permeation peptides, nuclear targeting signal peptides (NLS), polymers (for example, polymers with targeting groups, polymers with endosomal releasing agents, polymers with biodegradable properties, polymers with nucleic acid packing groups (by charge interactions, by hydrogen bonding interactions).

The above modifications can be made with the oligonucleotide agent with or without a linker (e.g., a cleavable linker), with or without a tether such as a long alkyl tether, with or without a spacer such as a PEG spacer, with or without a scaffold. The modifications can be made anywhere on the oligonucleotide agent, for example, at 5′-, 3′- or at internal positions.

In some embodiments, an oligonucleotide agent described herein includes a plurality of modifications. For example, an oligonucleotide agent such as an saRNA can include one or more backbone modifications, one or more 2′ ribose modifications, and also be conjugated to a ligand. For example, an oligonucleotide agent described herein can include phosphorthioate backbone modifications, and a 2′ ribose modification such as 2′-F, and further conjugated to a ligand such as a lipophilic ligand or folate.

Evaluation of Candidate Oligonucleotide Agents

One can evaluate a candidate single-stranded oligonucleotide agent, e.g., a modified candidate saRNA agent, for a selected property by exposing the agent or modified molecule and a control molecule to the appropriate conditions and evaluating for the presence of the selected property. For example, resistance to a degradant can be evaluated as follows. A candidate modified oligonucleotide agent, e.g., saRNA, can be exposed to degradative conditions, e.g., exposed to a milieu, which includes a degradative agent, e.g., a nuclease. For example, one can use a biological sample, e.g., one that is similar to a milieu, which might be encountered, in therapeutic use, e.g., blood or a cellular fraction, e.g., a cell-free homogenate or disrupted cells. The candidate and control can then be evaluated for resistance to degradation by any of a number of approaches. For example, the candidate and control could be labeled, preferably prior to exposure, with, e.g., a radioactive or enzymatic label, or a fluorescent label, such as Cy3 or Cy5. Control and modified oligonucleotide agents can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to increase gene expression. For example, a cell, e.g., a mammalian cell, such as a mouse or human cell, can be co-transfected with a plasmid expressing a fluorescent protein, e.g., GFP, and a candidate oligonucleotide agent homologous to the transcript encoding the fluorescent protein (see, e.g., WO 00/44914). For example, a modified oligonucleotide agent homologous to the GFP mRNA can be assayed for the ability to increase GFP expression by monitoring for a decrease in cell fluorescence, as compared to a control cell, in which the transfection did not include the candidate oligonucleotide agent, e.g., controls with no agent added and/or controls with a non-modified RNA added. Efficacy of the candidate agent on gene expression can be assessed by comparing cell fluorescence in the presence of the modified and unmodified oligonucleotide agent.

In an alternative functional assay, a candidate oligonucleotide agent homologous to an endogenous mouse gene, preferably a maternally expressed gene, such as c-mos, can be injected into an immature mouse oocyte to assess the ability of the agent to inhibit gene expression in vivo (see, e.g., WO 01/36646). A phenotype of the oocyte, e.g., the ability to maintain increase in metaphase II, can be monitored as an indicator that the agent is increasing expression. The effect of the modified agent on target RNA levels can be verified by Northern blot to assay for an increase in the level of target RNA, or by Western blot to assay for a decrease in the level of target protein, as compared to a negative control. Controls can include cells in which with no agent is added and/or cells in which a non-modified RNA is added.

Preferred Oligonucleotide Agents

Preferred single-stranded oligonucleotide agents have the following structure (see Formula 2 below):

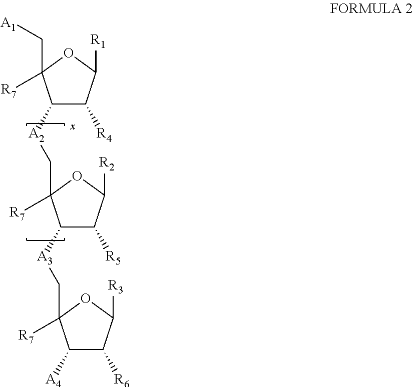

Referring to Formula 2 above, R¹, R², and R³ are each, independently, H, (i.e. abasic nucleotides), adenine, guanine, cytosine and uracil, inosine, thymine, xanthine, hypoxanthine, nubularine, tubercidine, isoguanisine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, 7-deazaguanine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases.

R⁴, R⁵, and R⁶ are each, independently, OR⁸, O(CH₂CH₂O)ₘCH₂CH₂OR⁸; O(CH₂)ₙR⁹; O(CH₂)ₙOR⁹, H; halo; NH₂; NHR⁸; N(R⁸)₂; NH(CH₂CH₂NH)ₘCH₂CH₂NHR⁹; NHC(O)R⁸; cyano; mercapto, SR⁸; alkyl-thio-alkyl; alkyl, aralkyl, cycloalkyl, aryl, heteroaryl, alkenyl, alkynyl, each of which may be optionally substituted with halo, hydroxy, oxo, nitro, haloalkyl, alkyl, alkaryl, aryl, aralkyl, alkoxy, aryloxy, amino, alkylamino, dialkylamino, heterocyclyl, arylamino, diaryl amino, heteroaryl amino, diheteroaryl amino, acylamino, alkylcarbamoyl, arylcarbamoyl, aminoalkyl, alkoxycarbonyl, carboxy, hydroxyalkyl, alkanesulfonyl, alkanesulfonamido, arenesulfonamido, aralkylsulfonamido, alkylcarbonyl, acyloxy, cyano, or ureido; or R⁴, R⁵, or R⁶ together combine with R⁷ to form an [—O—CH₂—] covalently bound bridge between the sugar 2′ and 4′ carbons.

A¹ is:

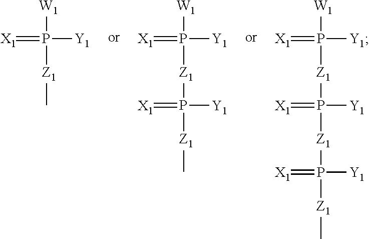

H; OH; OCH₃; W¹; an abasic nucleotide; or absent;

(a preferred A1, especially with regard to anti-sense strands, is chosen from 5′-monophosphate ((HO)₂(O)P—O-5′), 5′-diphosphate ((HO)₂(O)P—O—P(HO)(O)—O-5′), 5′-triphosphate ((HO)₂(O)P—O—(HO)(O)P—O—P(HO)(O)—O-5′), 5′-guanosine cap (7-methylated or non-methylated) (7m-G-O-5′-(HO)(O)P—O—(HO)(O)P—O—P(HO)(O)—O-5′), 5′-adenosine cap (Appp), and any modified or unmodified nucleotide cap structure (N—O-5′-(HO)(O)P—O—(HO)(O)P—O—P(HO)(O)—O-5′), 5′-monothiophosphate (phosphorothioate; (HO)₂(S)P—O-5′), 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g. RP(OH)(O)—O-5′-, (OH)₂(O)P-5′-CH₂—), 5′-alkyletherphosphonates (R=alkylether=methoxymethyl (MeOCH₂—), ethoxymethyl, etc., e.g. RP(OH)(O)—O-5′-)).

A² is:

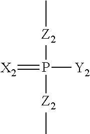

A³ is:

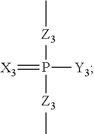

and

A⁴ is:

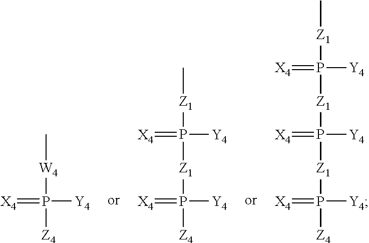

H; Z⁴; an inverted nucleotide; an abasic nucleotide; or absent.

W¹ is OH, (CH₂)ₙR¹⁰, (CH₂)ₙNHR¹⁰, (CH₂)ₙOR¹⁰, (CH₂)ₙSR¹⁰; O(CH₂)ₙR¹⁰; O(CH₂)ₙOR¹⁰, O(CH₂)ₙNR¹⁰, O(CH₂)ₙSR¹⁰; O(CH₂)ₙSS(CH₂)ₙOR¹⁰, O(CH₂)ₙC(O)OR¹⁰, NH(CH₂)ₙR¹⁰; NH(CH₂)ₙNR¹⁰; NH(CH₂)ₙOR¹⁰, NH(CH₂)ₙSR¹⁰; S(CH₂)ₙR¹⁰, S(CH₂)ₙNR¹⁰, S(CH₂)ₙOR¹⁰, S(CH₂)ₙSR¹⁰ O(CH₂CH₂O)ₘCH₂CH₂OR¹⁰; O(CH₂CH₂O)ₘCH₂CH₂NHR¹⁰, NH(CH₂CH₂NH)ₘCH₂CH₂NHR¹⁰; Q-R¹⁰, O-Q-R¹⁰ N-Q-R¹⁰, S-Q-R¹⁰ or —O—. W⁴ is O, CH₂, NH, or S.

X¹, X², X³, and X⁴ are each, independently, O or S.

Y¹, Y², Y³, and Y⁴ are each, independently, OH, O⁻, OR⁸, S, Se, BH₃⁻, H, NHR⁹, N(R⁹)₂ alkyl, cycloalkyl, aralkyl, aryl, or heteroaryl, each of which may be optionally substituted.

Z¹, Z², and Z³ are each independently O, CH₂, NH, or S, Z⁴ is OH, (CH₂)ₙR¹⁰, (CH₂)ₙNHR¹⁰, (CH₂)ₙ OR¹⁰, (CH₂)ₙ SR¹⁰; O(CH₂)ₙR¹⁰; O(CH₂)ₙOR¹⁰, O(CH₂)ₙNR¹⁰, O(CH₂)ₙSR¹⁰, O(CH₂)ₙSS(CH₂)ₙOR¹⁰, O(CH₂)ₙC(O)OR¹⁰; NH(CH₂)ₙR¹⁰; NH(CH₂)ₙNR¹⁰; NH(CH₂)ₙOR¹⁰, NH(CH₂)ₙSR¹⁰; S(CH₂)ₙR¹⁰, S(CH₂)ₙNR¹⁰, S(CH₂)ₙOR¹⁰, S(CH₂)ₙSR¹⁰ O(CH₂CH₂O)ₘCH₂CH₂OR¹⁰, O(CH₂CH₂O)ₘCH₂CH₂NHR¹⁰, NH(CH₂CH₂NH)ₘCH₂CH₂NHR¹⁰; Q-R¹⁰, O-Q-R¹⁰ N-Q-R¹⁰, S-Q-R¹⁰.

X is 5-100, chosen to comply with a length for an oligonucleotide agent described herein.

R⁷ is H; or is together combined with R⁴, R⁵, or R⁶ to form an [—O—CH₂—] covalently bound bridge between the sugar 2′ and 4′ carbons.

R⁸ is alkyl, cycloalkyl, aryl, aralkyl, heterocyclyl, heteroaryl, amino acid, or sugar; R⁹ is NH₂, alkylamino, dialkylamino, heterocyclyl, arylamino, diaryl amino, heteroaryl amino, diheteroaryl amino, or amino acid; and R¹⁰ is H; fluorophore (pyrene, TAMRA, fluorescein, Cy3 or Cy5 dyes); sulfur, silicon, boron or ester protecting group; intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic carriers (cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino; alkyl, cycloalkyl, aryl, aralkyl, heteroaryl; radiolabelled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles); or an oligonucleotide agent. M is 0-1,000,000, and n is 0-20. Q is a spacer selected from the group consisting of abasic sugar, amide, carboxy, oxyamine, oxyimine, thioether, disulfide, thiourea, sulfonamide, or morpholino, biotin or fluorescein reagents.

Preferred oligonucleotide agents in which the entire phosphate group has been replaced have the following structure (see Formula 3 below):

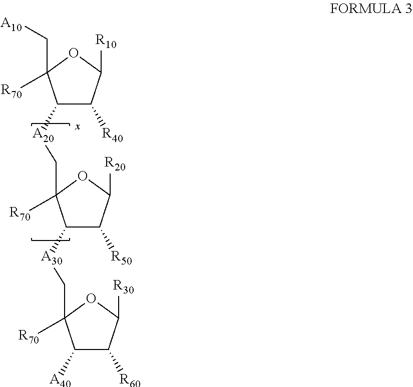

Referring to Formula 3, A¹⁰-A⁴⁰ is L-G-L; A¹⁰ and/or A⁴⁰ may be absent, in which L is a linker, wherein one or both L may be present or absent and is selected from the group consisting of CH₂(CH₂)_{g}; N(CH₂)_{g}; O(CH₂)_{g}; S(CH₂)_{g}. G is a functional group selected from the group consisting of siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

R¹⁰, R²⁰, and R³⁰ are each, independently, H, (i.e. abasic nucleotides), adenine, guanine, cytosine and uracil, inosine, thymine, xanthine, hypoxanthine, nubularine, tubercidine, isoguanisine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, 7-deazaguanine, N6, N6-dimethyladenine, 2,6-diaminopurine, 5-amino-allyl-uracil, N3-methyluracil substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases.

R⁴⁰, R⁵⁰, and R⁶⁰ are each, independently, OR⁸, O(CH₂CH₂O)ₘCH₂CH₂OR⁸; O(CH₂)ₙR⁹; O(CH₂)ₙOR⁹, H; halo; NH₂; NHR⁸; N(R⁸)₂; NH(CH₂CH₂NH)ₘCH₂CH₂R⁹; NHC(O)R⁸; cyano; mercapto, SR⁷; alkyl-thio-alkyl; alkyl, aralkyl, cycloalkyl, aryl, heteroaryl, alkenyl, alkynyl, each of which may be optionally substituted with halo, hydroxy, oxo, nitro, haloalkyl, alkyl, alkaryl, aryl, aralkyl, alkoxy, aryloxy, amino, alkylamino, dialkylamino, heterocyclyl, arylamino, diaryl amino, heteroaryl amino, diheteroaryl amino, acylamino, alkylcarbamoyl, arylcarbamoyl, aminoalkyl, alkoxycarbonyl, carboxy, hydroxyalkyl, alkanesulfonyl, alkanesulfonamido, arenesulfonamido, aralkylsulfonamido, alkylcarbonyl, acyloxy, cyano, and ureido groups; or R⁴⁰, R⁵⁰, or R⁶⁰ together combine with R⁷⁰ to form an [—O—CH₂—] covalently bound bridge between the sugar 2′ and 4′ carbons.

X is 5-100 or chosen to comply with a length for an oligonucleotide agent described herein.

R⁷⁰ is H; or is together combined with R⁴⁰, R⁵⁰, or R⁶⁰ to form an [—O—CH₂—] covalently bound bridge between the sugar 2′ and 4′ carbons.

R⁸ is alkyl, cycloalkyl, aryl, aralkyl, heterocyclyl, heteroaryl, amino acid, or sugar; and R⁹ is NH₂, alkylamino, dialkylamino, heterocyclyl, arylamino, diaryl amino, heteroaryl amino, diheteroaryl amino, or amino acid. M is 0-1,000,000, n is 0-20, and g is 0-2.

Preferred nucleoside surrogates have the following structure (see Formula 4 below):

SLR¹⁰⁰-(M-SLR²⁰⁰)ₓ-M-SLR³⁰⁰ FORMULA 4

S is a nucleoside surrogate selected from the group consisting of mophilino, cyclobutyl, pyrrolidine and peptide nucleic acid. L is a linker and is selected from the group consisting of CH₂(CH₂)_{g}; N(CH₂)_{g}; O(CH₂)_{g}; S(CH₂)_{g}; —C(O)(CH₂)ₙ— or may be absent. M is an amide bond; sulfonamide; sulfinate; phosphate group; modified phosphate group as described herein; or may be absent.

R¹⁰⁰, R²⁰⁰, and R³⁰⁰ are each, independently, H (i.e., abasic nucleotides), adenine, guanine, cytosine and uracil, inosine, thymine, xanthine, hypoxanthine, nubularine, tubercidine, isoguanisine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, 7-deazaguanine, N6, N6-dimethyladenine, 2,6-diaminopurine, 5-amino-allyl-uracil, N3-methyluracil substituted 1,2,4-triazoles, 2-pyridinones, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases.

X is 5-100, or chosen to comply with a length for an oligonucleotide agent described herein; and g is 0-2.

An oligonucleotide agent featured in the invention, e.g., an saRNA, can include an internucleotide linkage (e.g., the chiral phosphorothioate linkage) useful for increasing nuclease resistance. In addition, or in the alternative, an oligonucleotide agent can include a ribose mimic for increased nuclease resistance. Exemplary internucleotide linkages and ribose mimics for increased nuclease resistance are described in co-owned PCT Application No. PCT/US2004/07070 filed on Mar. 8, 2004.

An oligonucleotide agent featured in the invention, e.g., an saRNA, can include a ligand-conjugated monomer subunit and monomers for oligonucleotide synthesis. Exemplary monomers are described in co-owned U.S. application Ser. No. 10/916,185, filed on Aug. 10, 2004. In some preferred embodiments, the ligand is conjugated to the saRNA via a pyrrolidone moiety. In some embodiments, the conjugate is linked to the pyrrolidone moiety via a linker moiety.

An oligonucleotide agent featured in the invention, e.g., an saRNA, can have a ZXY structure, such as is described in co-owned PCT Application No. PCT/US2004/07070 filed on Mar. 8, 2004.

An oligonucleotide agent featured in the invention, e.g., an saRNA, can be complexed with an amphipathic moiety. Exemplary amphipathic moieties for use with oligonucleotide agents are described in co-owned PCT Application No. PCT/US2004/07070 filed on Mar. 8, 2004.

In another embodiment, the oligonucleotide agent featured in the invention, e.g., an saRNA, can be complexed to a delivery agent that features a modular complex. The complex can include a carrier agent linked to one or more of (preferably two or more, more preferably all three of): (a) a condensing agent (e.g., an agent capable of attracting, e.g., binding, a nucleic acid, e.g., through ionic or electrostatic interactions); (b) a fusogenic agent (e.g., an agent capable of fusing and/or being transported through a cell membrane); and (c) a targeting group, e.g., a cell or tissue targeting agent, e.g., a lectin, glycoprotein, lipid or protein, e.g., an antibody, that binds to a specified cell type. oligonucleotide agents complexed to a delivery agent are described in co-owned PCT Application No. PCT/US2004/07070 filed on Mar. 8, 2004. In some preferred embodiments, the delivery agent includes one or more of TETA-5LAP, DLinDMA, and Analogs with PEGS and Cholesterol or another lipophilic moiety such as a fatty acid described herein.

### Enhanced Nuclease Resistance

An saRNA described herein can have enhanced resistance to nucleases.

For increased nuclease resistance and/or binding affinity to the target, an oligonucleotide agent, e.g., the oligonucleotide agent, can include, for example, 2′-modified ribose units and/or phosphorothioate linkages. E.g., the 2′ hydroxyl group (OH) can be modified or replaced with a number of different “oxy” or “deoxy” substituents.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; amine, G-AMINE and aminoalkoxy, O(CH₂)ₙAMINE, (e.g., AMINE=NH₂; alkylamino, dialkylamino, heterocyclyl amino, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e. deoxyribose sugars); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl amino, arylamino, diaryl amino, heteroaryl amino, or diheteroaryl amino), —NHC(O)R(R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality.

Preferred substitutents are 2′-methoxyethyl, 2′-OCH3, 2′-O-allyl, 2′-C-allyl, and 2′-fluoro.

One way to increase resistance is to identify cleavage sites and modify such sites to inhibit cleavage, as described in co-owned U.S. Application No. 60/559,917, filed on May 4, 2004. For example, the dinucleotides 5′-UA-3′,5′-UG-3′,5′-CA-3′,5′-UU-3′, or 5′-CC-3′ can serve as cleavage sites. Enhanced nuclease resistance can therefore be achieved by modifying the 5′ nucleotide, resulting, for example, in at least one 5′-uridine-adenine-3′ (5′-UA-3′) dinucleotide wherein the uridine is a 2′-modified nucleotide; at least one 5′-uridine-guanine-3′ (5′-UG-3′) dinucleotide, wherein the 5′-uridine is a 2′-modified nucleotide; at least one 5′-cytidine-adenine-3′ (5′-CA-3′) dinucleotide, wherein the 5′-cytidine is a 2′-modified nucleotide; at least one 5′-uridine-uridine-3′ (5′-UU-3′) dinucleotide, wherein the 5′-uridine is a 2′-modified nucleotide; or at least one 5′-cytidine-cytidine-3′ (5′-CC-3′) dinucleotide, wherein the 5′-cytidine is a 2′-modified nucleotide. The oligonucleotide agent can include at least 2, at least 3, at least 4 or at least 5 of such dinucleotides. In certain embodiments, all the pyrimidines of an oligonucleotide agent carry a 2′-modification, and the oligonucleotide agent therefore has enhanced resistance to endonucleases.

To maximize nuclease resistance, the 2′ modifications can be used in combination with one or more phosphate linker modifications (e.g., phosphorothioate). The so-called “chimeric” oligonucleotides are those that contain two or more different modifications.

The inclusion of furanose sugars in the oligonucleotide backbone can also decrease endonucleolytic cleavage. An oligonucleotide agent can be further modified by including a 3′ cationic group, or by inverting the nucleoside at the 3′-terminus with a 3′-3′ linkage. In another alternative, the 3′-terminus can be blocked with an aminoalkyl group, e.g., a 3′ C5-aminoalkyl dT. Other 3′ conjugates can inhibit 3′-5′ exonucleolytic cleavage. While not being bound by theory, a 3′ conjugate, such as naproxen or ibuprofen, may inhibit exonucleolytic cleavage by sterically blocking the exonuclease from binding to the 3′-end of oligonucleotide. Even small alkyl chains, aryl groups, or heterocyclic conjugates or modified sugars (D-ribose, deoxyribose, glucose etc.) can block 3′-5′-exonucleases.

Similarly, 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′-end of oligonucleotide. Even small alkyl chains, aryl groups, or heterocyclic conjugates or modified sugars (D-ribose, deoxyribose, glucose etc.) can block 3′-5′-exonucleases.

Thus, an oligonucleotide agent can include modifications so as to inhibit degradation, e.g., by nucleases, e.g., endonucleases or exonucleases, found in the body of a subject. These monomers are referred to herein as NRMs, or Nuclease Resistance promoting Monomers, the corresponding modifications as NRM modifications. In many cases these modifications will modulate other properties of the oligonucleotide agent as well, e.g., the ability to interact with a protein, e.g., a transport protein, e.g., serum albumin, or a member of the RISC, or the ability of the oligonucleotide agent to form a duplex with another sequence, e.g., a target molecule, such as an miRNA or pre-miRNA.

One or more different NRM modifications can be introduced into an oligonucleotide agent or into a sequence of an oligonucleotide agent. An NRM modification can be used more than once in a sequence or in an oligonucleotide agent.

NRM modifications include some which can be placed only at the terminus and others which can go at any position. Some NRM modifications that can inhibit hybridization are preferably used only in terminal regions, and more preferably not at the cleavage site or in the cleavage region of the oligonucleotide agent.

Such modifications can be introduced into the terminal regions, e.g., at the terminal position or with 2, 3, 4, or 5 positions of the terminus, of a sequence which targets or a sequence which does not target a sequence in the subject.

### Delivery of Single-Stranded Oligonucleotide Agents to Tissues and Cells

### Formulation

The single-stranded oligonucleotide agents described herein can be formulated for administration to a subject.

For ease of exposition, the formulations, compositions, and methods in this section are discussed largely with regard to unmodified oligonucleotide agents. It should be understood, however, that these formulations, compositions, and methods can be practiced with other oligonucleotide agents, e.g., modified oligonucleotide agents, and such practice is within the invention.

A formulated oligonucleotide agent featured in the invention, e.g., an saRNA composition can assume a variety of states. In some examples, the composition is at least partially crystalline, uniformly crystalline, and/or anhydrous (e.g., less than 80, 50, 30, 20, or 10% water). In another example, the oligonucleotide agent is in an aqueous phase, e.g., in a solution that includes water, this form being the preferred form for administration via inhalation.

The aqueous phase or the crystalline compositions can be incorporated into a delivery vehicle, e.g., a liposome (particularly for the aqueous phase), or a particle (e.g., a microparticle as can be appropriate for a crystalline composition). Generally, the oligonucleotide agent composition is formulated in a manner that is compatible with the intended method of administration. In some preferred embodiments, an oligonucleotide agent described herein is incorporated into a liposome delivery vehicle. Exemplary liposome delivery vehicles includes those containing one or more of TETA-5LAP, DLinDMA, and Analogs with PEGS and Cholesterol. In some embodiments, the PEG lipid has the following structure:

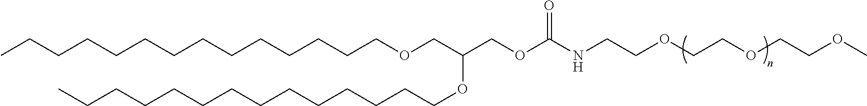

wherein the repeating PEG moiety has an average molecular weight of 2000 with n value between 42 and 47.

An oligonucleotide agent preparation can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, the oligonucleotide agent preparation includes a second oligonucleotide agent, e.g., a second agent that can down-regulate expression of a second gene. Still other preparations can include at least three, five, ten, twenty, fifty, or a hundred or more different oligonucleotide species. In some embodiments, the agents are directed to the same target nucleic acid but different target sequences. In another embodiment, each oligonucleotide agent is directed to a different target. In one embodiment the oligonucleotide agent preparation includes a double stranded RNA that targets a non-coding sequence of a target gene.

### Treatment Methods and Routes of Delivery

A composition that includes an agent featured in the invention, e.g., an agent that targets a non-coding sequence of a target gene can be delivered to a subject by a variety of routes. Exemplary routes include inhalation, intrathecal, parenchymal, intravenous, nasal, oral, and ocular delivery.

An oligonucleotide featured in the invention, e.g., an saRNA, can be incorporated into pharmaceutical compositions suitable for administration. For example, compositions can include one or more oligonucleotide agents and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions featured in the invention may be administered in a number of ways depending upon whether local or systemic treatment is desired and upon the area to be treated. Administration may be topical (including ophthalmic, intranasal, transdermal, intrapulmonary), oral or parenteral. Parenteral administration includes intravenous drip, subcutaneous, intraperitoneal or intramuscular injection, or intrathecal or intraventricular administration.

In general, delivery of an agent featured in the invention directs the agent to the site of infection in a subject. The preferred means of delivery is through local administration directly to the site of infection, or by systemic administration, e.g. parental administration.

Formulations for direct injection and parenteral administration are well known in the art. Such formulations may include sterile aqueous solutions which may also contain buffers, diluents and other suitable additives. For intravenous use, the total concentration of solutes should be controlled to render the preparation isotonic.

Administration of Oligonucleotide Agents A patient who has been diagnosed with a disorder characterized by under expression of a target gene can be treated by administration of an oligonucleotide agent described herein, thereby alleviating the symptoms associated with the under expression of a target gene. Similarly, a human who has or is at risk for developing a disorder characterized by under expression of a target gene can be treated by the administration of an oligonucleotide agent that targets the a non-coding sequence of a target gene (e.g., a promoter region of the target gene).

The oligonucleotide agents described herein can be administered systemically, e.g., orally or by intramuscular injection or by intravenous injection, in admixture with a pharmaceutically acceptable carrier adapted for the route of administration. An oligonucleotide agent can include a delivery vehicle, such as liposomes, for administration to a subject, carriers and diluents and their salts, and/or can be present in pharmaceutically acceptable formulations. Methods for the delivery of nucleic acid molecules are described in Akhtar et al., Trends in Cell Bio. 2:139, 1992; Delivery Strategies for Antisense Oligonucleotide Therapeutics, ed. Akhtar, 1995; Maurer et al., Mol. Membr. Biol., 16:129, 1999; Hofland and Huang, Handb. Exp. Pharmacol. 137:165, 1999; and Lee et al., ACS Symp. Ser. 752:184, 2000, all of which are incorporated herein by reference. Beigelman et al., U.S. Pat. No. 6,395,713 and Sullivan et al., PCT WO 94/02595 further describe the general methods for delivery of nucleic acid molecules. Nucleic acid molecules can be administered to cells by a variety of methods known to those of skill in the art, including, but not restricted to, encapsulation in liposomes, by iontophoresis, or by incorporation into other vehicles, such as hydrogels, cyclodextrins (see for example Gonzalez et al., Bioconjugate Chem. 10:1068, 1999), biodegradable nanocapsules, and bioadhesive microspheres, or by proteinaceous vectors (O'Hare and Normand, International PCT Publication No. WO 00/53722).

In the present methods, the oligonucleotide agent can be administered to the subject either as a naked oligonucleotide agent, in conjunction with a delivery reagent, or as a recombinant plasmid or viral vector which expresses the oligonucleotide agent. Preferably, the oligonucleotide agent is administered as a naked oligonucleotide agent.

An oligonucleotide agent featured in the invention can be administered to the subject by any means suitable for delivering the agent to the cells of the tissue at or near the area of unwanted target nucleic acid expression (e.g., target miRNA or pre-miRNA expression). For example, an oligonucleotide agent that targets miR-122 can be delivered directly to the liver, or can be conjugated to a molecule that targets the liver. Exemplary delivery methods include administration by gene gun, electroporation, or other suitable parenteral administration route.

Suitable enteral administration routes include oral delivery.

Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection, intra-retinal injection, or sub-retinal injection); subcutaneous injection or deposition including subcutaneous infusion (such as by osmotic pumps); direct application to the area at or near the site of neovascularization, for example by a catheter or other placement device (e.g., a retinal pellet or an implant comprising a porous, non-porous, or gelatinous material).

An oligonucleotide agent featured in the invention can be delivered using an intraocular implant. Such implants can be biodegradable and/or biocompatible implants, or may be non-biodegradable implants. The implants may be permeable or impermeable to the active agent, and may be inserted into a chamber of the eye, such as the anterior or posterior chambers, or may be implanted in the sclera, transchoroidal space, or an avascularized region exterior to the vitreous. In a preferred embodiment, the implant may be positioned over an avascular region, such as on the sclera, so as to allow for transscleral diffusion of the drug to the desired site of treatment, e.g., the intraocular space and macula of the eye. Furthermore, the site of transscleral diffusion is preferably in proximity to the macula.

An oligonucleotide agent featured in the invention can also be administered topically, for example, by patch or by direct application to the eye, or by iontophoresis. Ointments, sprays, or droppable liquids can be delivered by ocular delivery systems known in the art such as applicators or eyedroppers. The compositions can be administered directly to the surface of the eye or to the interior of the eyelid. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers.

An oligonucleotide agent featured in the invention may be provided in sustained release compositions, such as those described in, for example, U.S. Pat. Nos. 5,672,659 and 5,595,760. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained release composition may be appropriate.

An oligonucleotide agent can be injected into the interior of the eye, such as with a needle or other delivery device.

An oligonucleotide agent featured in the invention can be administered in a single dose or in multiple doses. Where the administration of the oligonucleotide agent is by infusion, the infusion can be a single sustained dose or can be delivered by multiple infusions. Injection of the agent can be directly into the tissue at or near the site of aberrant or unwanted target gene expression (e.g., aberrant or unwanted miRNA or pre-miRNA expression). Multiple injections of the agent can be made into the tissue at or near the site.

Dosage levels on the order of about 1 μg/kg to 100 mg/kg of body weight per administration are useful in the treatment of a disease. One skilled in the art can also readily determine an appropriate dosage regimen for administering an oligonucleotide agent, e.g., a supermir, to a given subject. For example, the oligonucleotide agent can be administered to the subject once, e.g., as a single injection or deposition at or near the site on unwanted target nucleic acid expression. Alternatively, the oligonucleotide agent can be administered once or twice daily to a subject for a period of from about three to about twenty-eight days, more preferably from about seven to about ten days. In a preferred dosage regimen, the oligonucleotide agent is injected at or near a site of unwanted target nucleic acid expression once a day for seven days. Where a dosage regimen comprises multiple administrations, it is understood that the effective amount of an oligonucleotide agent administered to the subject can include the total amount of agent administered over the entire dosage regimen. One skilled in the art will appreciate that the exact individual dosages may be adjusted somewhat depending on a variety of factors, including the specific oligonucleotide agent being administered, the time of administration, the route of administration, the nature of the formulation, the rate of excretion, the particular disorder being treated, the severity of the disorder, the pharmacodynamics of the oligonucleotide agent, and the age, sex, weight, and general health of the patient. Wide variations in the necessary dosage level are to be expected in view of the differing efficiencies of the various routes of administration. For instance, oral administration generally would be expected to require higher dosage levels than administration by intravenous or intravitreal injection. Variations in these dosage levels can be adjusted using standard empirical routines of optimization, which are well-known in the art. The precise therapeutically effective dosage levels and patterns are preferably determined by the attending physician in consideration of the above-identified factors.

In addition to treating pre-existing diseases or disorders, oligonucleotide agents featured in the invention can be administered prophylactically in order to prevent or slow the onset of a particular disease or disorder. In prophylactic applications, an oligonucleotide agent is administered to a patient susceptible to or otherwise at risk of a particular disorder, such as disorder associated with underexpression of a target gene.

The oligonucleotide agents featured by the invention are preferably formulated as pharmaceutical compositions prior to administering to a subject, according to techniques known in the art. Pharmaceutical compositions featured in the present invention are characterized as being at least sterile and pyrogen-free. As used herein, “pharmaceutical formulations” include formulations for human and veterinary use. Methods for preparing pharmaceutical compositions are within the skill in the art, for example as described in Remington's Pharmaceutical Science, 18th ed., Mack Publishing Company, Easton, Pa. (1990), and The Science and Practice of Pharmacy, 2003, Gennaro et al., the entire disclosures of which are herein incorporated by reference.

The present pharmaceutical formulations include an oligonucleotide agent featured in the invention (e.g., 0.1 to 90% by weight), or a physiologically acceptable salt thereof, mixed with a physiologically acceptable carrier medium. Preferred physiologically acceptable carrier media are water, buffered water, normal saline, 0.4% saline, 0.3% glycine, hyaluronic acid and the like.

Pharmaceutical compositions featured in the invention can also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). Pharmaceutical compositions can be packaged for use in liquid form, or can be lyophilized.

For solid compositions, conventional non-toxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can include any of the carriers and excipients listed above and 10-95%, preferably 25%-75%, of one or more single-stranded oligonucleotide agents featured in the invention.

By “pharmaceutically acceptable formulation” is meant a composition or formulation that allows for the effective distribution of the nucleic acid molecules of the instant invention in the physical location most suitable for their desired activity. Non-limiting examples of agents suitable for formulation with the nucleic acid molecules of the instant invention include: P-glycoprotein inhibitors (such as PluronicP85), which can enhance entry of drugs into the CNS (Jolliet-Riant and Tillement, Fundam. Clin. Pharmacol. 13:16, 1999); biodegradable polymers, such as poly (DL-lactide-coglycolide) microspheres for sustained release delivery. Other non-limiting examples of delivery strategies for the nucleic acid molecules featured in the instant invention include material described in Boado et al., J. Pharm. Sci. 87:1308, 1998; Tyler et al., FEBS Lett. 421:280, 1999; Pardridge et al., PNAS USA. 92:5592, 1995; Boado, Adv. Drug Delivery Rev. 15:73, 1995; Aldrian-Herrada et al., Nucleic Acids Res. 26:4910, 1998; and Tyler et al., PNAS USA 96:7053, 1999.

The invention also features the use of a composition that includes surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al., Chem. Rev. 95:2601, 1995; Ishiwata et al., Chem. Phare. Bull. 43:1005, 1995).

Such liposomes have been shown to accumulate selectively in tumors, presumably by extravasation and capture in the neovascularized target tissues (Lasic et al., Science 267:1275, 1995; Oku et al., Biochim. Biophys. Acta 1238:86, 1995). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 42:24864, 1995; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also features compositions prepared for storage or administration that include a pharmaceutically effective amount of the desired oligonucleotides in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

Alternatively, certain oligonucleotide agents featured in the instant invention can be expressed within cells from eukaryotic promoters (e.g., Izant and Weintraub, Science 229:345, 1985; McGarry and Lindquist, Proc. Natl. Acad. Sci. USA 83:399, 1986; Scanlon et al., Proc. Natl. Acad. Sci. USA 88:10591, 1991; Kashani-Sabet et al., Antisense Res. Dev. 2:3, 1992; Dropulic et al., J. Virol. 66:1432, 1992; Weerasinghe et al., J. Virol. 65:5531, 1991; Ojwang et al., Proc. Natl. Acad. Sci. USA 89:10802, 1992; Chen et al., Nucleic Acids Res. 20:4581, 1992; Sarver et al., Science 247:1222, 1990; Thompson et al., Nucleic Acids Res. 23:2259, 1995; Good et al., Gene Therapy 4:45, 1997). Those skilled in the art realize that any nucleic acid can be expressed in eukaryotic cells from the appropriate DNA/RNA vector. The activity of such nucleic acids can be augmented by their release from the primary transcript by an enzymatic nucleic acid (Draper et al., PCT WO 93/23569, and Sullivan et al., PCT WO 94/02595; Ohkawa et al., Nucleic Acids Symp. Ser. 27:156, 1992; Taira et al., Nucleic Acids Res. 19:5125, 1991; Ventura et al., Nucleic Acids Res. 21:3249, 1993; Chowrira et al., J. Biol. Chem. 269:25856, 1994).

In another aspect of the invention, oligonucleotide agents of the present invention can be expressed from transcription units (see for example Couture et al., Trends in Genetics 12:510, 1996) inserted into DNA or RNA vectors. The recombinant vectors can be DNA plasmids or viral vectors. Oligonucleotide agent-expressing viral vectors can be constructed based on, but not limited to, adeno-associated virus, retrovirus, adenovirus, or alphavirus. In another embodiment, pol III based constructs are used to express nucleic acid molecules of the invention (see for example Thompson, U.S. Pats. Nos. 5,902,880 and 6,146,886). The recombinant vectors capable of expressing the oligonucleotide agents can be delivered as described above, and can persist in target cells. Alternatively, viral vectors can be used that provide for transient expression of nucleic acid molecules. Such vectors can be repeatedly administered as necessary. Once expressed, the oligonucleotide agent interacts with the target RNA (e.g., miRNA or pre-miRNA) and inhibits miRNA activity. In a preferred embodiment, the oligonucleotide agent forms a duplex with the target miRNA, which prevents the miRNA from binding to its target mRNA, which results in increased translation of the target mRNA. Delivery of oligonucleotide agent-expressing vectors can be systemic, such as by intravenous or intra-muscular administration, by administration to target cells ex-planted from a subject followed by reintroduction into the subject, or by any other means that would allow for introduction into the desired target cell (for a review see Couture et al., Trends in Genetics 12:510, 1996).

The term “therapeutically effective amount” is the amount present in the composition that is needed to provide the desired level of drug in the subject to be treated to give the anticipated physiological response.

The term “physiologically effective amount” is that amount delivered to a subject to give the desired palliative or curative effect.

The term “pharmaceutically acceptable carrier” means that the carrier can be taken into the subject with no significant adverse toxicological effects on the subject.

The term “co-administration” refers to administering to a subject two or more single-stranded oligonucleotide agents. The agents can be contained in a single pharmaceutical composition and be administered at the same time, or the agents can be contained in separate formulation and administered serially to a subject. So long as the two agents can be detected in the subject at the same time, the two agents are said to be co-administered.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. A preferred group of carbohydrates includes lactose, threhalose, raffinose maltodextrins, and mannitol. Suitable polypeptides include aspartame. Amino acids include alanine and glycine, with glycine being preferred.

Suitable pH adjusters or buffers include organic salts prepared from organic acids and bases, such as sodium citrate, sodium ascorbate, and the like; sodium citrate is preferred.

Dosage. An oligonucleotide agent featured in the invention, e.g., a supermir, can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of agent per kg of bodyweight. The unit dose, for example, can be administered by injection (e.g., intravenous or intramuscular, intrathecally, or directly into an organ), inhalation, or a topical application.

Delivery of an agent directly to an organ (e.g., directly to the liver) can be at a dosage on the order of about 0.00001 mg to about 3 mg per organ, or preferably about 0.0001-0.001 mg per organ, about 0.03-3.0 mg per organ, about 0.1-3.0 mg per organ or about 0.3-3.0 mg per organ.

The dosage can be an amount effective to treat or prevent a disease or disorder.

In one embodiment, the unit dose is administered less frequently than once a day, e.g., less than every 2, 4, 8 or 30 days. In another embodiment, the unit dose is not administered with a frequency (e.g., not a regular frequency). For example, the unit dose may be administered a single time. Because oligonucleotide agent-mediated silencing can persist for several days after administering the oligonucleotide agent composition, in many instances, it is possible to administer the composition with a frequency of less than once per day, or, for some instances, only once for the entire therapeutic regimen.

In one embodiment, a subject is administered an initial dose, and one or more maintenance doses of an oligonucleotide agent. The maintenance dose or doses are generally lower than the initial dose, e.g., one-half less of the initial dose. A maintenance regimen can include treating the subject with a dose or doses ranging from 0.01 μg to 75 mg/kg of body weight per day, e.g., 70, 60, 50, 40, 30, 20, 10, 5, 2, 1, 0.5, 0.1, 0.05, 0.01, 0.005, 0.001, or 0.0005 mg per kg of bodyweight per day. The maintenance doses are preferably administered no more than once every 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

Following successful treatment, it may be desirable to have the patient undergo maintenance therapy to prevent the recurrence of the disease state, wherein the compound of the invention is administered in maintenance doses, ranging from 0.01 μg to 100 g per kg of body weight (see U.S. Pat. No. 6,107,094).

The concentration of the oligonucleotide agent composition is an amount sufficient to be effective in treating or preventing a disorder or to regulate a physiological condition in humans. The concentration or amount of oligonucleotide agent administered will depend on the parameters determined for the agent and the method of administration, e.g. direct administration to the eye. For example, eye formulations tend to require much lower concentrations of some ingredients in order to avoid irritation or burning of the ocular tissues. It is sometimes desirable to dilute an oral formulation up to 10-100 times in order to provide a suitable ocular formulation.

Certain factors may influence the dosage required to effectively treat a subject, including but not limited to the severity of the disease or disorder, previous treatments, the general health and/or age of the subject, and other diseases present. It will also be appreciated that the effective dosage of the oligonucleotide agent used for treatment may increase or decrease over the course of a particular treatment. Changes in dosage may result and become apparent from the results of diagnostic assays. For example, the subject can be monitored after administering an composition containing the oligonucleotide agent. Based on information from the monitoring, an additional amount of the composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC₅₀s found to be effective in in vitro and in vivo animal models.

The invention is further illustrated by the following examples, which should not be construed as further limiting.

### EXAMPLES

### Example 1

### High Affinity Sugar-Base Modifications

Oligonucleotide Agents Containing High Affinity Nucleoside Modifications

High Affinity Sugar-Base Modifications

At least one of the listed nucleotide in Exemplification 2 is present in the oligonucleotide agent shown in Exemplification 1.

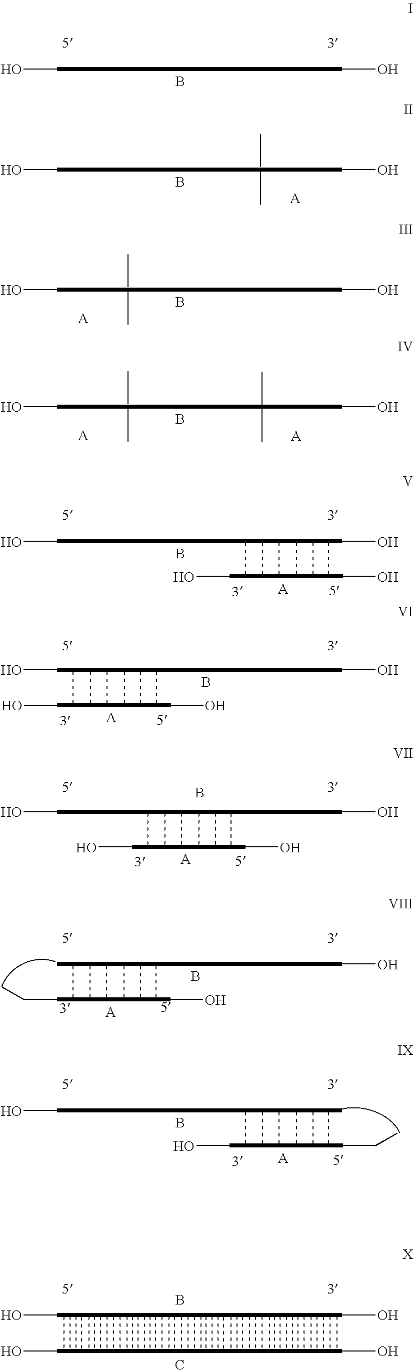

Exemplification 1. Oligonucleotide agent designs. I: Oligonucleotide agent; II: Oligonucleotide agent with 3′-ribosugar and phosphate (2-8 nucleotide in length); III: Oligonucleotide agent with 5′-ribosugar and phosphate (2-8 nucleotide in length); IV: Oligonucleotide agent with 3′ and 5′-ribosugar and phosphate (2-8 nucleotide in length); V: Oligonucleotide agent with 3′-end partial duplex with oligoribonucleotide (4-8 nucleotide in length); VI: Oligonucleotide agent with 5′-end partial duplex with oligoribonucleotide (4-8 nucleotide in length); VII: Oligonucleotide agent with internal partial duplex with oligoribonucleotide (4-8 nucleotide in length); VII: Oligonucleotide agent with 5′-end partial hairpin with oligoribonucleotide (4-8 nucleotide in length); IX: Oligonucleotide agent with 3′-end partial hairpin with oligoribonucleotide (4-8 nucleotide in length); X: Oligonucleotide agent with inactivated complementary antisense strand. Segment A indicates oligoribonucleotide with phosphate backbone; segment B indicates Oligonucleotide agent and segment C indicates inactivated antisense strand complementary to a Oligonucleotide agent.

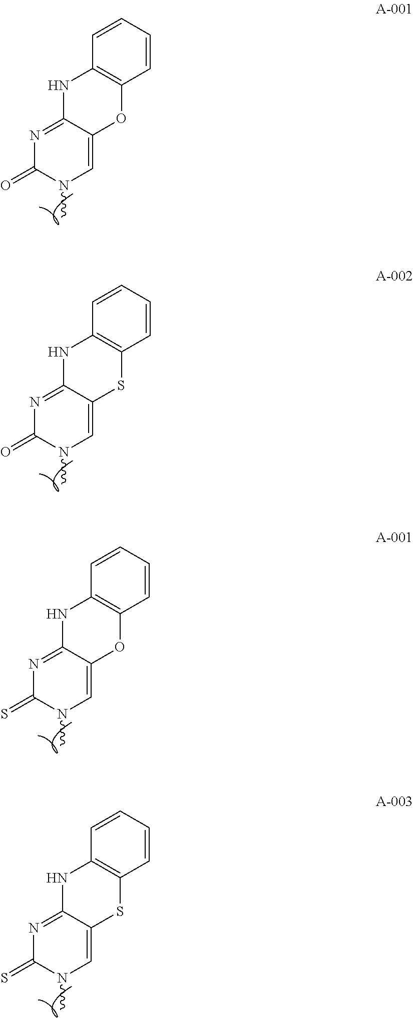

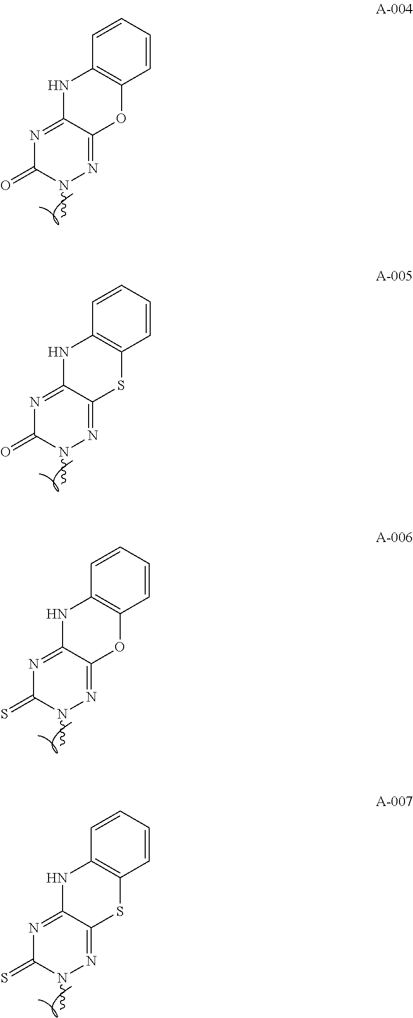

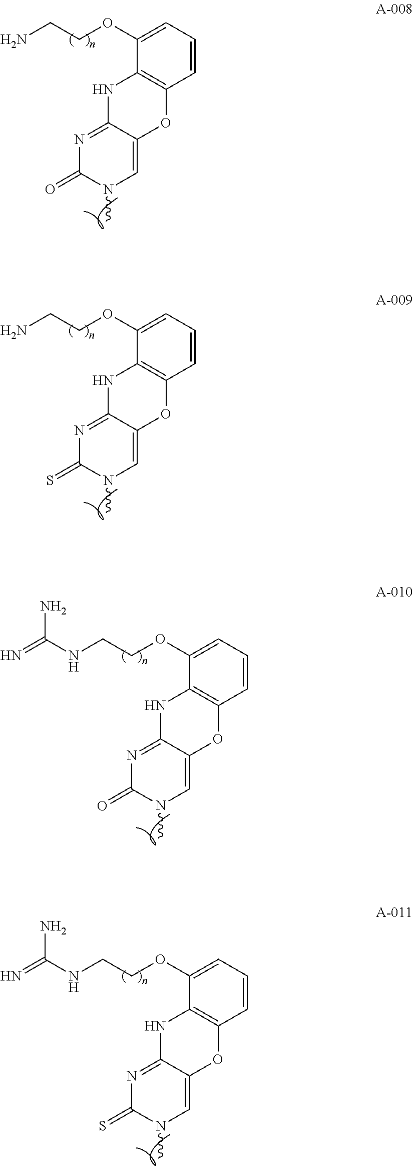

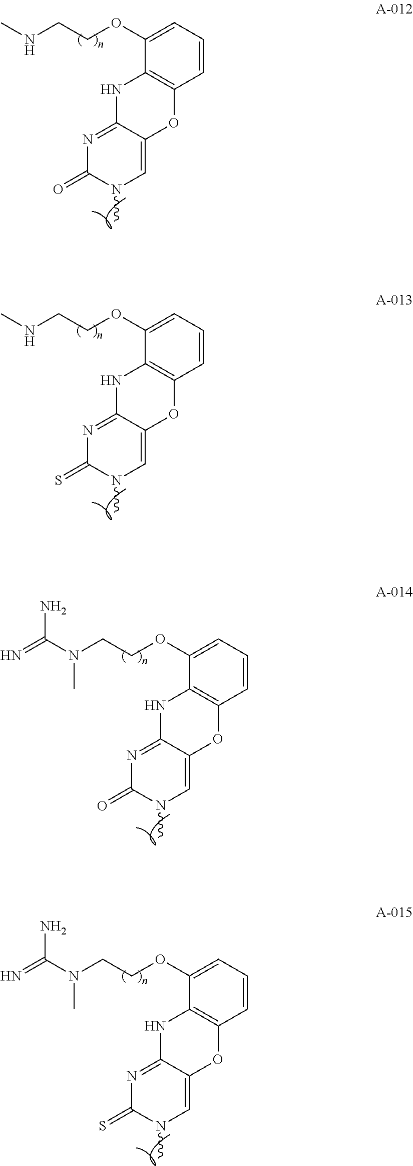

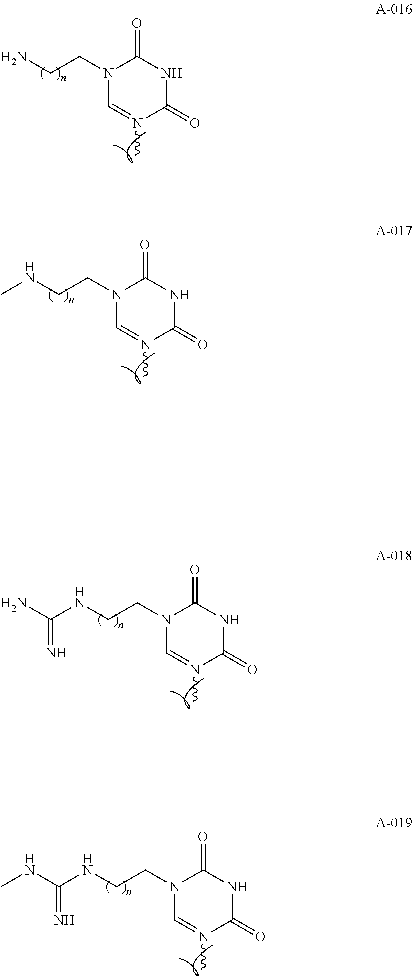

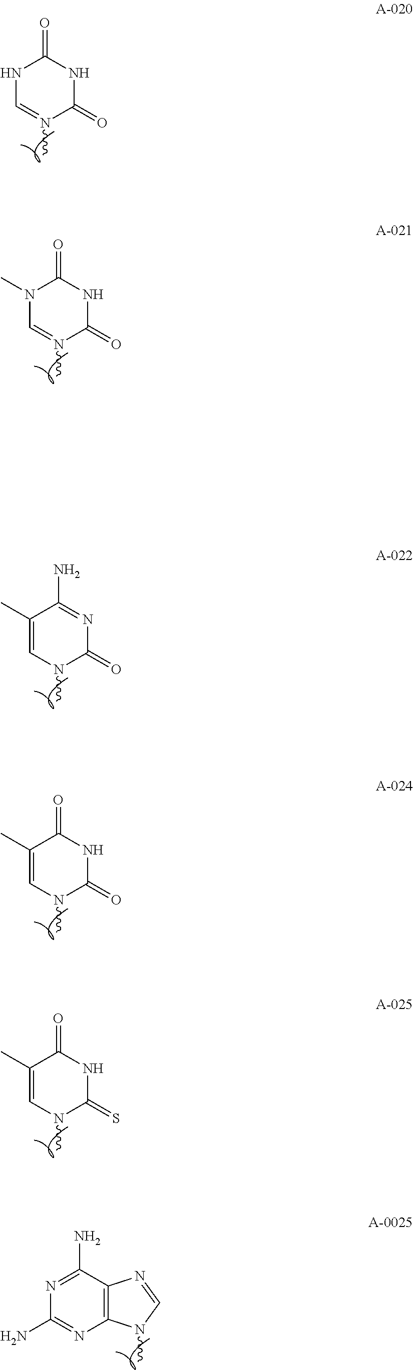

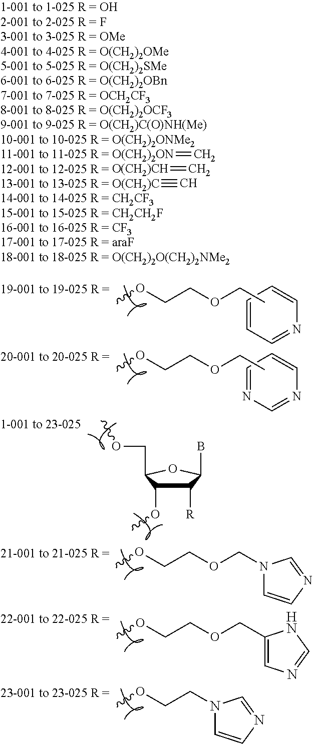

Exemplification 2. Compound 1-001 to 23-025 represents oligonucleotide agents with corresponding nucleoside modification. Lower case ‘n’=0-11 and uppercase A=1 to 24.

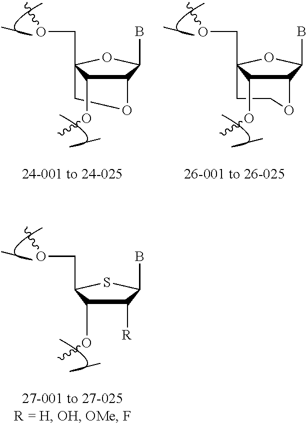

Exemplification 3. Sugar modifications. 24: LNA; 25: ENA and 26: 4′-Thio. B is from A-001 to A-025 of Exemplification 2.

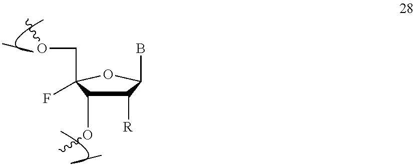

Exemplification 3. 4′-Fluoro sugar modification

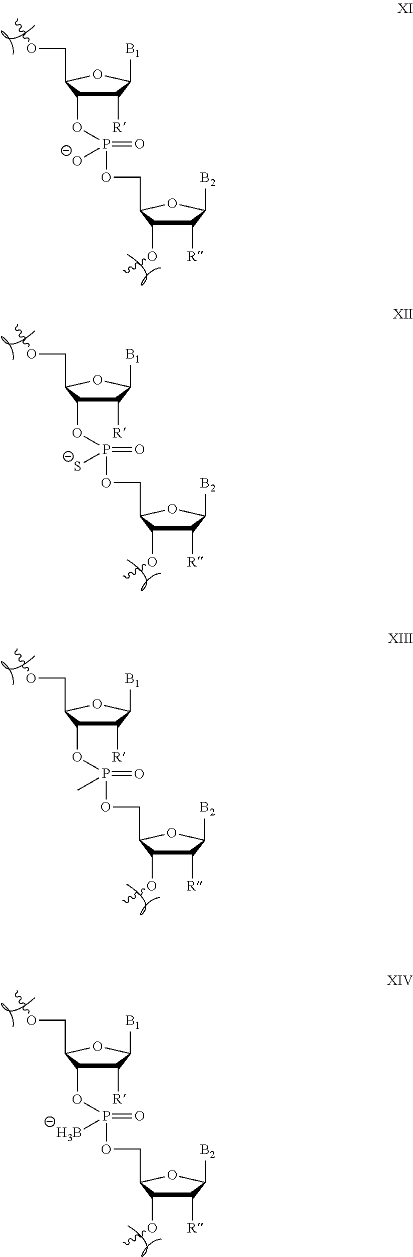

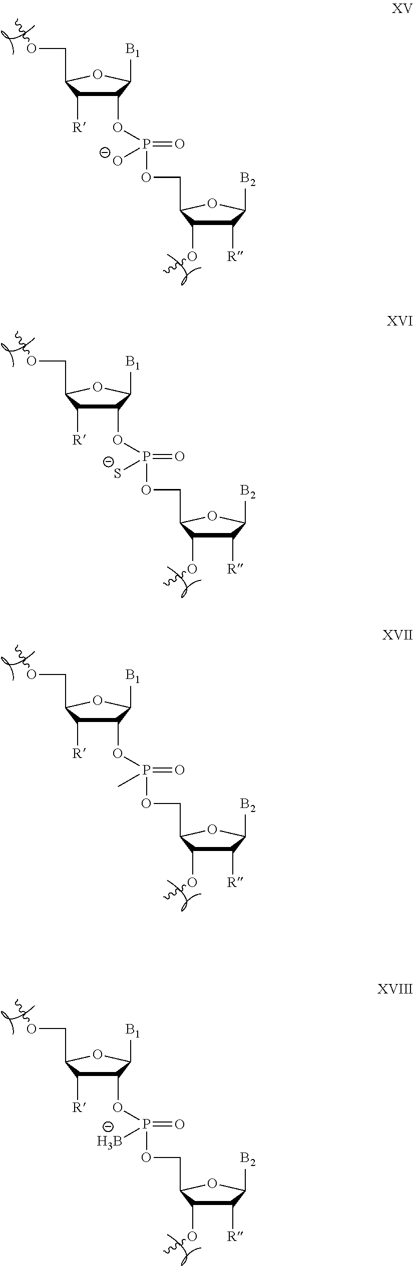

Exemplification 4. Backbone linkages. XI: 3′-5′ Phosphate; XII: 3′,5′ phosphorothioate; XIII 3′,5′ methylphosphonate; XIV: 3′,5′ boranophosphate; Xv: 2′-5′Phosphate; XVI: 2′,5′ phosphorothioate; XVII 2′,5′ methylphosphonate; XVII: 2′,5′ boranophosphate

### Example 2

### High Affinity Oligonucleotides Containing 2-amino-2′-deoxy-2′-fluoro-adenosine

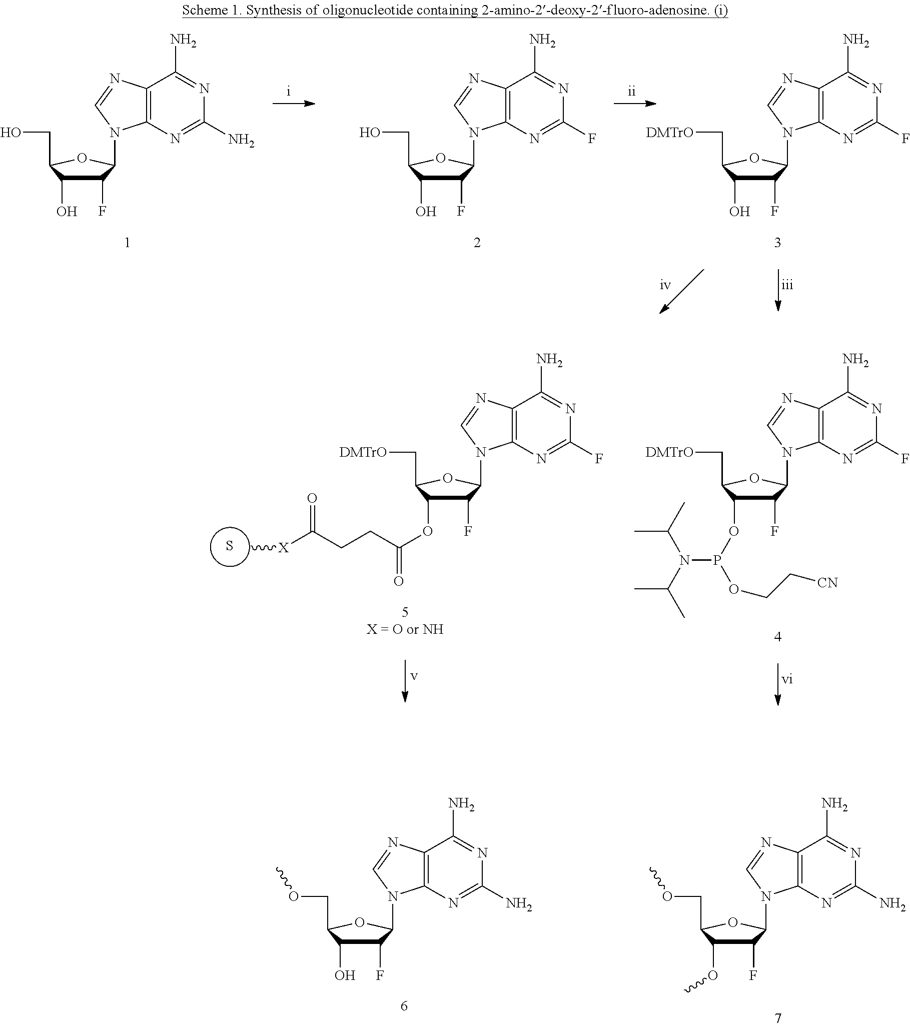

Step 1 Compound 2: A 2 L polyethylene bottle was equipped with a magnetic stirrer, thermometer, dry ice/acetone bath and a stream of argon gas. Anhydrous pyridine (500 mL) was added and the solution was cooled to −20° C. To this was added 70% hydrogen fluoride in pyridine (400 mL). 2′-fluoro-2,6-diaminopurine riboside (1, 90 g, 0.317 mol) was dissolved in the solution. Tert-butylnitrite (75 mL, 0.63 mol) was added in one portion and the reaction was stirred at 6-12° C. until the reaction was complete as judged by TLC (3 h). Sodium bicarbonate (2000 g) was suspended with manual stirring in water (2 L) in a 20 L bucket. The reaction solution was slowly poured (to allow for evolution of carbon dioxide) into the aqueous layer with vigorous stirring. The resulting solution was extracted with ethyl acetate (3×500 mL). The organic layers were combined and concentrated to a solid. The solid was mostly dissolved in methanol (300 mL) at reflux. The solution was cooled in a ice water bath and the resulting solid was collected, rinsed with methanol (2×50 mL) and dried under vacuum (1 mm Hg, 25° C., 24 h) to give 55 g of compound 2 as a dark gold solid. The mother liquor was concentrated and purified by column chromatography to give an additional 15.1 g of product for a total of 70.1 g (77%).

Step 2 Compound 3: 2,2′-Difluoro-2′-deoxyadenosine (2, 70 g, 0.244 mol) and dimethoxytrityl chloride (91.0 g, 0.268 mol) were dissolved in anhydrous pyridine (700 mL) and stirred at ambient temperature for 3 h. The reaction was quenched by the addition of methanol (50 mL) and then concentrated under reduced pressure to an oil. The residue was partitioned between ethyl acetate (1 L) and sat'd sodium bicarbonate (1 L mL). The aqueous layer was extracted with ethyl acetate (500 mL) once more and the combined extracts were concentrated under reduced pressure. The resulting solid was purified by crystallization from hexane-ethyl acetate (1:1) to give a light brown solid 3 (120.1 g, 83%).

Step 3 Compound 4: 5′-O-(4,4′-Dimethoxytrityl)-2,2′-difluoro-2′-deoxyadenosine (3, 90 g, 0.153 mol), 2-cyanoethyl tetraisopropylphosphorodiamidite (55.0 g, 0.183 mol), diisopropylamine tetrazolide (17.0 g, 0.1 mol) were dissolved in anhydrous dichloromethane (1 L) and allowed to stir at ambient temperature under an argon atmosphere for 16 h. The reaction was concentrated under reduced pressure to a thin oil and then directly applied to a silica gel column (200 g). The product was eluted with ethyl acetate-triethylamine (99:1). The appropriate fractions were combined, concentrated under reduced pressure, coevaporated with anhydrous acetonitrile and dried (1 mm Hg, 25° C., 24 h) to 109.1 g (90%) of light yellow foam of compound 4.

Step 4 Compound 5: 5′-O-(4,4′-Dimethoxytrityl)-2,2′-difluoro-2′-deoxyadenosine (4, 10 g, 16.9 mmol), dimethylaminopyridine (0.32 g, 2.6 mmol) and succinic anhydride (3.4 g, 34 mmol) were dissolved in anhydrous pyridine (50 mL) and stirred at ambient temperature under an argon atmosphere for 6 h. The reaction was quenched by the addition of water (20 mL) and then concentrated under reduced pressure to an oil. The oil was purified by column chromatography, eluted with methanol-dichloromethane-triethylamine (7:92:1). The appropriate fractions were collected and evaporated to give the product as a light yellow solid (10.4 g, 78%) of the corresponding succinate. The succinate is subsequently attached to solid support as reported in the literature to obtain the desired solid support 5.

Step 5 Compound 6: Oligonucleotide containing 2-Amino-2′-fluoro-adenosine at the 3′-end is synthesized starting from the solid support 5 according to the standard solid phase oligonucleotide synthesis and deprotection protocols. (Ref: WO00012563 and WO01002608). Oligonucleotide with phosphodiester and phosphorothioate backbone are prepared as reported by Ross et al. (WO00012563 and WO01002608)

Step 6 Compound 7: Oligonucleotide containing 2-amino-adenosine with 2′-deoxy-2′-fluoro sugar modification is synthesized and characterized as reported by Manoharan and Cook (Ref: WO01002608).

### Example 3

### High Affinity Oligonucleotides Containing Other 2-amino-adenosine Modifications

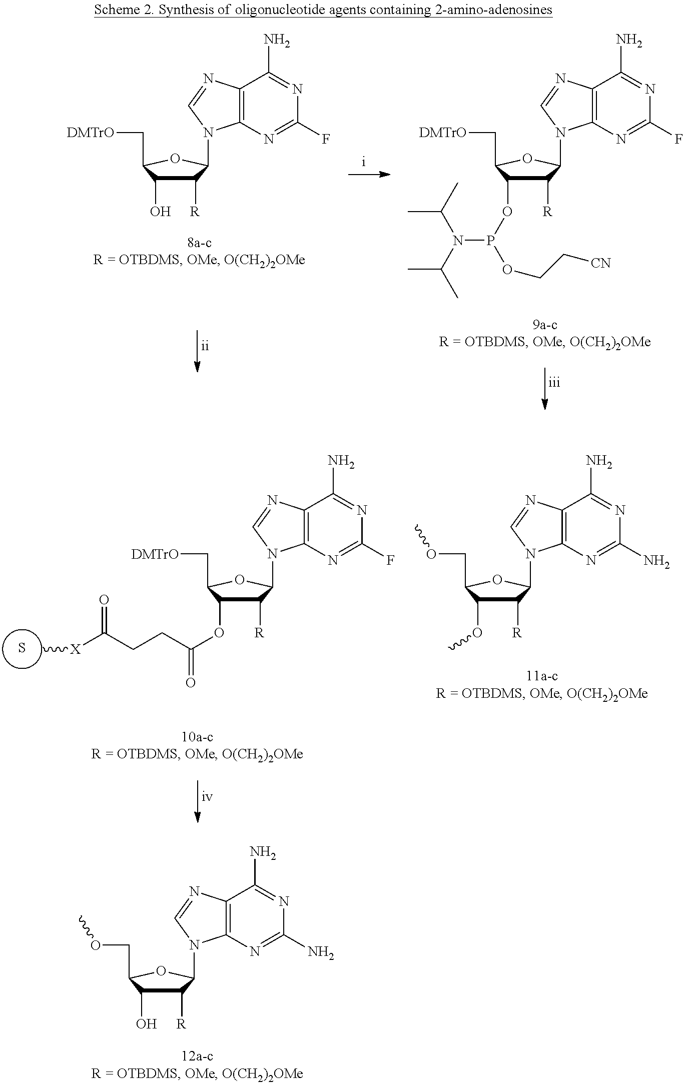

Compounds 8a-c are prepared as reported by Manoharan and Cook (Ref: WO01002608). Oligonucleotides with 2-amino-adenosine base modification (11a-c and 12a-c) are prepared as described in Example 1.

### Example 4

### High Affinity Oligonucleotides Containing High Affinity Phenoxazine and Thiophenoxazine

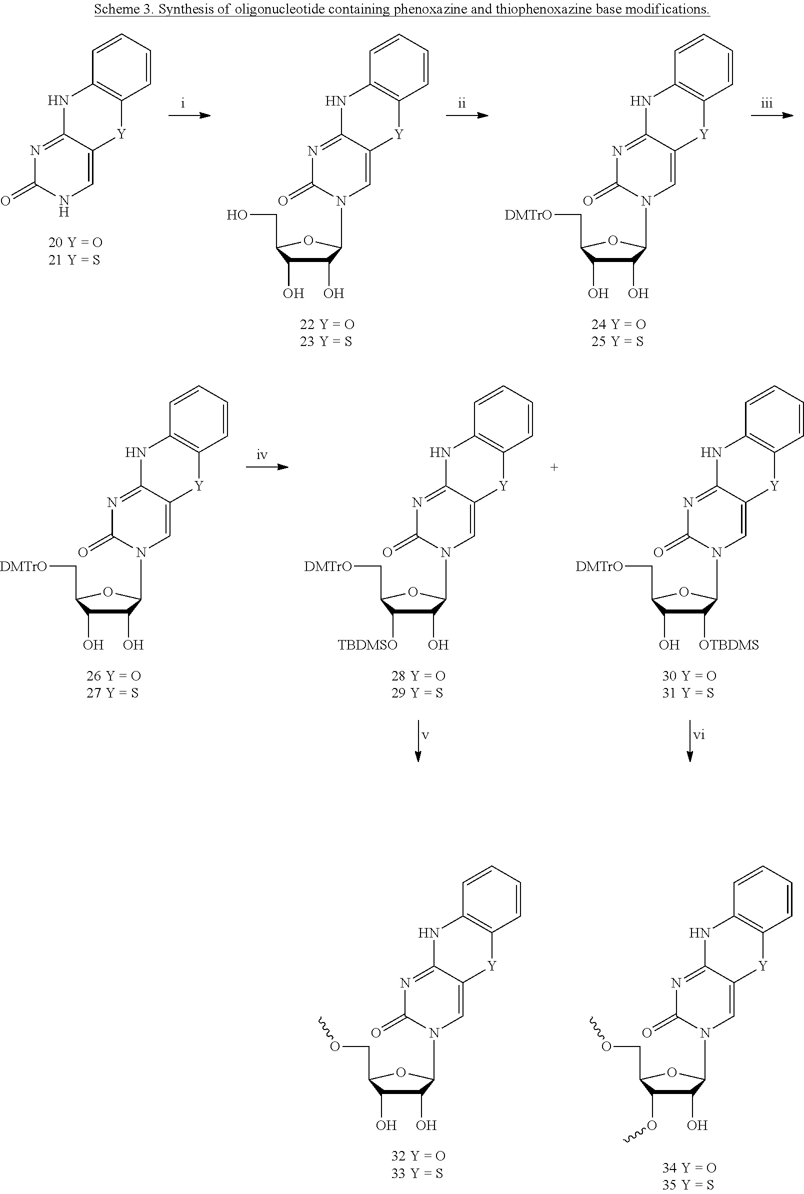

Step 1 Compounds 22 and 23: Compounds 20 and 21 are prepared according to the literature procedure (Sandin, Peter; Lincoln, Per; Brown, Tom; Wilhelmsson, L. Marcus. Nature Protocols, 2007, 2(3), 615-623). Nucleosides 22 and 23 are obtained respectively from 20 and 21 according to the procedures reported by Rajeev and Broom (Organic Lett., 2000, 2, 3595). Compounds 28 to 31 are obtained from respectively from 22 and 23 according to procedure reported by Xia et al. (ACS Chem. Biol., 2006, 1, 176). Oligonucleotides 32 to 35 are synthesized from corresponding precursors 28 to 31 as described in Example 1 and as reported by Sandin et al. (Nature Protocols, 2007, 2(3), 615-623).

### Example 5

### High Affinity Oligonucleotides with G-Clamp Modification

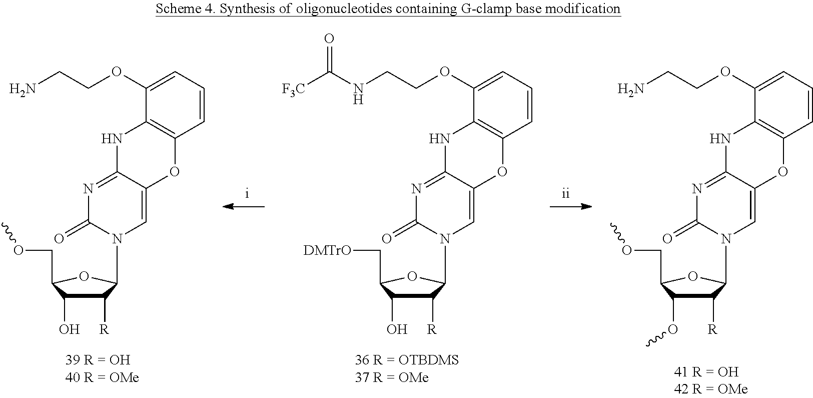

Compound 36 and 37 are prepared as reported by Holmes et al., Nucleic Acids Res., 2003, 31, 2759. Oligonucleotides 39 to 42 are obtained from corresponding precursors 36 and 37 as described in Examples 3 and 1.

### Example 6

### High Affinity Oligonucleotides Containing 2′-OMe and 2′-deoxy-2′-F Sugar Modified Phenoxazine Nucleosides

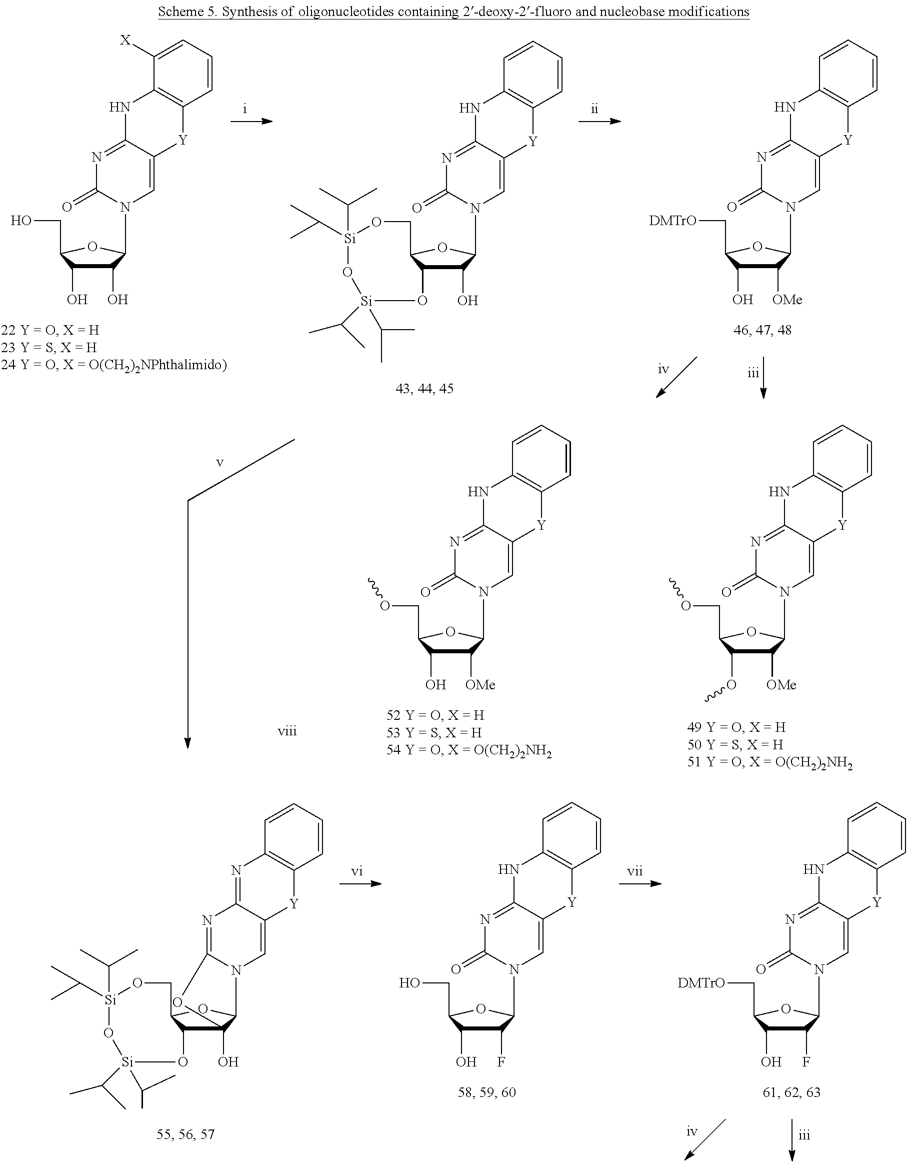

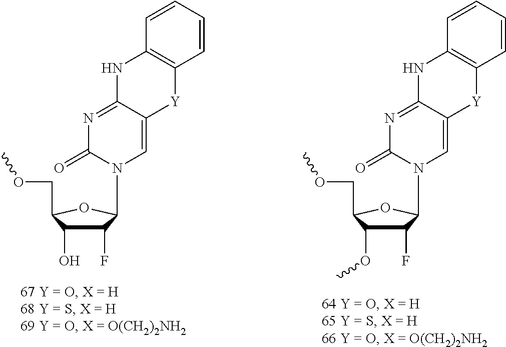

Compounds 46, 47 and 48 are prepared according to reported procedures (Holmes et al., Nucleic Acids Res., 2003, 31, 2759). Oligonucleotides 49 to 54 are obtained from corresponding precursors 47, 47 and 48 as described in Example 1

Compounds 58, 59 and 60 are prepared according to reported procedures (Shi et al., Bioorg. Med. Chem., 2005, 13, 1641). Oligonucleotides 64 to 69 are obtained from corresponding precursors 58, 59 and 60 as described in Example 1

### Example 6

### High Affinity Oligonucleotides Containing Pseudouridine Base Modifications

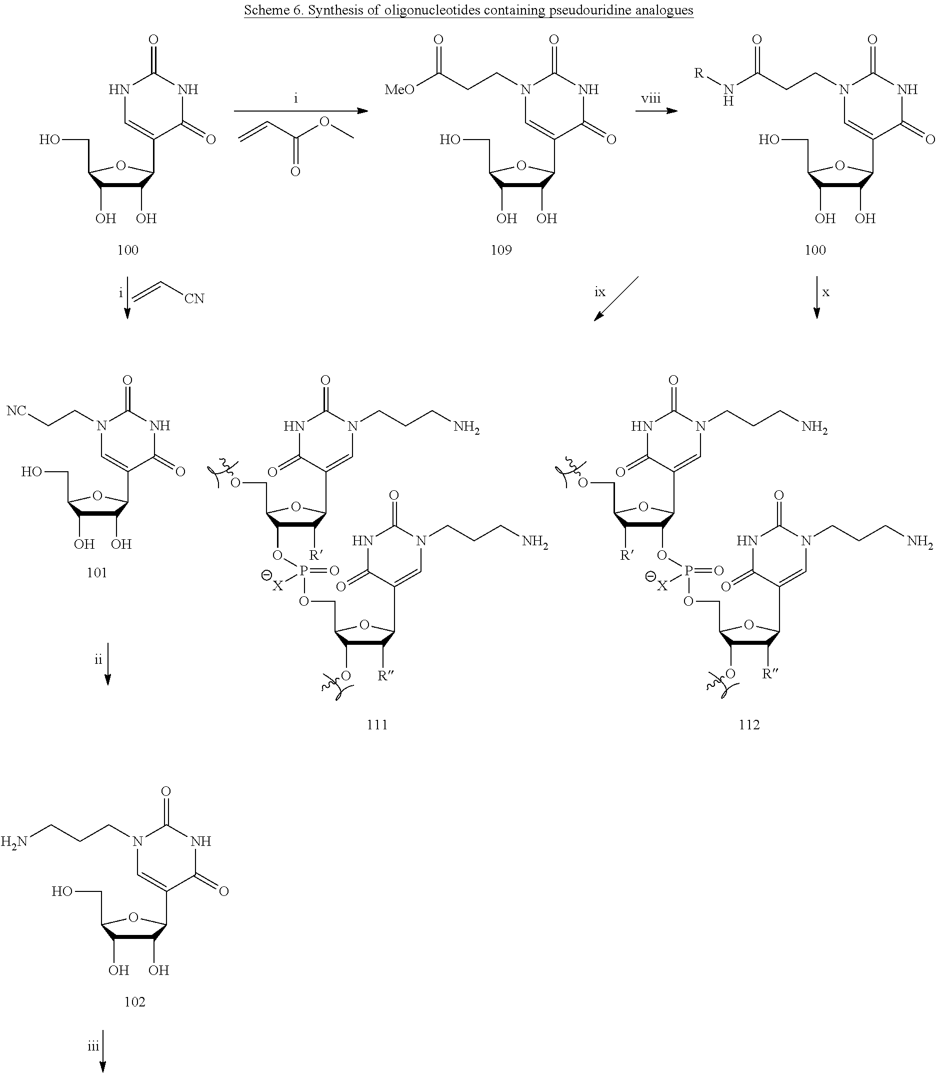

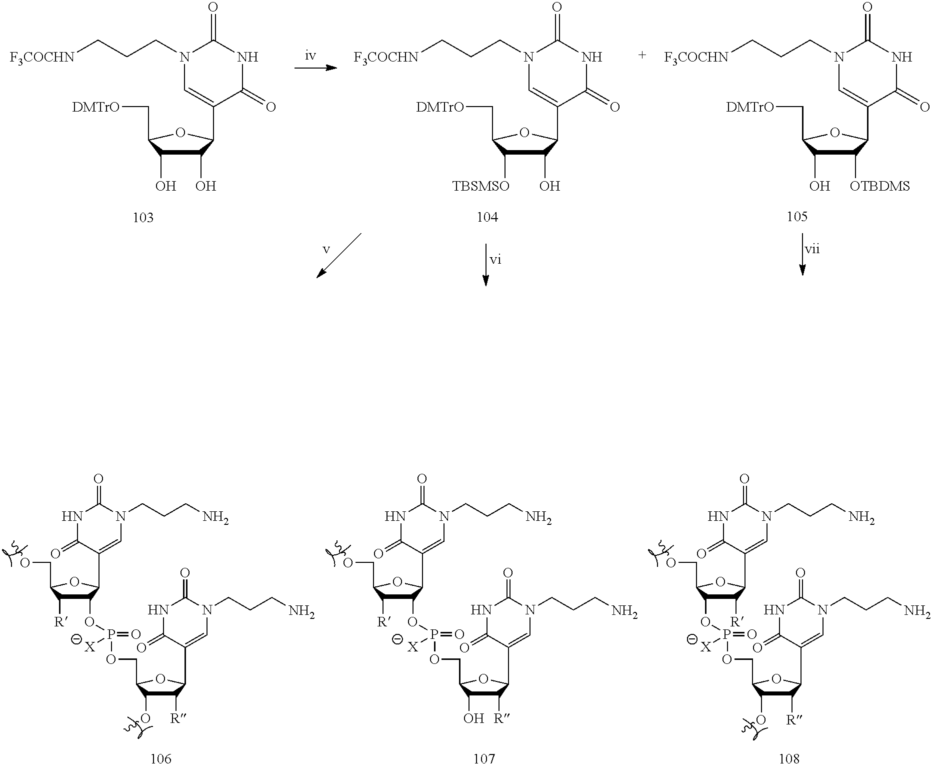

### Example 7

### Synthesis of Oligonucleotide Agents

Step 1. Oligonucleotide Synthesis

All oligonucleotides were synthesized on an AKTAoligopilot synthesizer or on an ABI 394 DNA/RNA synthesizer. Commercially available controlled pore glass solid supports (rU-CPG, 2′-O-methyl modified rA-CPG and 2′-O-methyl modified rG-CPG from Prime Synthesis) or the in-house synthesized solid support hydroxyprolinol-cholesterol-CPG (described in patent xxxx) were used for the synthesis. RNA phosphoramidites and 2′-O-methyl modified RNA phosphoramidites with standard protecting groups (5′-O-dimethoxytrityl-N6-benzoyl-2′-t-butyldimethylsilyl-adenosine-3′-O—N,N′-diisopropyl-2-cyanoethylphosphoramidite, 5′-O-dimethoxytrityl-N4-acetyl-2′-t-butyldimethylsilyl-cytidine-3′-O—N,N′-diisopropyl-2-cyanoethylphosphoramidite, 5′-O-dimethoxytrityl-N2-isobutryl-2′-t-butyldimethylsilyl-guanosine-3′-O—N,N′-diisopropyl-2-cyanoethylphosphoramidite, 5′-O-dimethoxytrityl-2′-t-butyldimethylsilyl-uridine-3′-O—N,N′-diisopropyl-2-cyanoethylphosphoramidite, 5′-O-dimethoxytrityl-N6-benzoyl-2′-O-methyl-adenosine-3′-O—N,N′-diisopropyl-2-cyanoethylphosphoramidite, 5′-O-dimethoxytrityl-N4-acetyl-2′-O-methyl-cytidine-3′-O—N,N′-diisopropyl-2-cyanoethylphosphoramidite, 5′-O-dimethoxytrityl-N2-isobutryl-2′-O-methyl-guanosine-3′-O—N,N′-diisopropyl-2-cyanoethylphosphoramidite, 5′-O-dimethoxytrityl-2′-O-methyl-uridine-3′-O—N,N′-diisopropyl-2-cyanoethylphosphoramidite and 5′-O-dimethoxytrityl-2′-deoxy-thymidine-3′-O—N,N′-diisopropyl-2-cyanoethylphosphoramidite) were obtained from Pierce Nucleic Acids Technologies and ChemGenes Research. The Quasar 570 phosphoramidite was obtained from Biosearch Technologies. The 5′-O-dimethoxytrityl-2′-t-butyldimethylsilyl-inosine-3′-O—N,N′-diisopropyl-2-cyanoethylphosphoramidite was obtained from ChemGenes Research. The 5′-O-dimethoxytrityl-2′-t-butyldimethylsilyl-(2,4)-difluorotolyl-3′-O—N,N′-diisopropyl-2-cyanoethylphosphoramidite (DFT-phosphoramidite) and the 5′-O-dimethoxytrityl-2′-t-butyldimethylsilyl-9-(2-aminoethoxy)-phenoxazine-3′-O—N,N′-diisopropyl-2-cyanoethylphosphoramidite (G-clamp phosphoramidite) were synthesized in house.

For the syntheses on AKTAoligopilot synthesizer, all phosphoramidites were used at a concentration of 0.2 M in CH₃CN except for guanosine and 2′-O-methyl-uridine, which were used at 0.2 M concentration in 10% THF/CH₃CN (v/v). Coupling/recycling time of 16 minutes was used for all phosphoramidite couplings. The activator was 5-ethyl-thio-tetrazole (0.75 M, American International Chemicals). For the PO-oxidation, 50 mM iodine in water/pyridine (10:90 v/v) was used and for the PS-oxidation 2% PADS (GL Synthesis) in 2,6-lutidine/CH₃CN (1:1 v/v) was used. For the syntheses on ABI 394 DNA/RNA synthesizer, all phosphoramidites, including DFT and G-clamp phosphoramidites were used at a concentration of 0.15 M in CH₃CN except for 2′-O-methyl-uridine, which was used at 0.15 M concentration in 10% THF/CH₃CN (v/v). Coupling time of 10 minutes was used for all phosphoramidite couplings. The activator was 5-ethyl-thio-tetrazole (0.25 M, Glen Research). For the PO-oxidation, 20 mM iodine in water/pyridine (Glen Research) was used and for the PS-oxidation 0.1M DDTT (AM Chemicals) in pyridine was used. Coupling of the Quasar 570 phosphoramidite was carried out on the ABI DNA/RNA synthesizer. The Quasar 570 phosphoramidite was used at a concentration of 0.1M in CH₃CN with a coupling time of 10 mins. The activator was 5-ethyl-thio-tetrazole (0.25 M, Glen Research) and 0.1M DDTT (AM Chemicals) in pyridine was used for PS oxidation.

Step 2. Deprotection of Oligonucleotides

A. Sequences Synthesized on the AKTAoligopilot Synthesizer

After completion of synthesis, the support was transferred to a 100 mL glass bottle (VWR). The oligonucleotide was cleaved from the support with simultaneous deprotection of base and phosphate groups with 40 mL of a 40% aq. methyl amine (Aldrich) 90 mins at 45° C. The bottle was cooled briefly on ice and then the methylamine was filtered into a new 500 mL bottle. The CPG was washed three times with 40 mL portions of DMSO. The mixture was then cooled on dry ice.

In order to remove the tert-butyldimethylsilyl (TBDMS) groups at the 2′ position, 60 mL triethylamine trihydrofluoride (Et3N—HF) was added to the above mixture. The mixture was heated at 40° C. for 60 minutes. The reaction was then quenched with 220 mL of 50 mM sodium acetate (pH 5.5) and stored in the freezer until purification.

B. Sequences Synthesized on the ABI DNA/RNA Synthesizer

After completion of synthesis, the support was transferred to a 15 mL tube (VWR). The oligonucleotide was cleaved from the support with simultaneous deprotection of base and phosphate groups with 7 mL of a 40% aq. methyl amine (Aldrich) 15 mins at 65° C. The bottle was cooled briefly on ice and then the methylamine was filtered into a 100 mL bottle (VWR). The CPG was washed three times with 7 mL portions of DMSO. The mixture was then cooled on dry ice.

In order to remove the tert-butyldimethylsilyl (TBDMS) groups at the 2′ position, 10.5 mL triethylamine trihydrofluoride (Et3N—HF) was added to the above mixture. The mixture was heated at 60° C. for 15 minutes. The reaction was then quenched with 38.5 mL of 50 mM sodium acetate (pH 5.5) and stored in the freezer until purification.

Step 3. Quantitation of Crude Oligonucleotides

For all samples, a 10 μL aliquot was diluted with 990 μL of deionised nuclease free water (1.0 mL) and the absorbance reading at 260 nm was obtained.

Step 4. Purification of Oligonucleotides

### (a) Unconjugated Oligonucleotides

The unconjugated crude oligonucleotides were first analyzed by HPLC (Dionex PA 100). The buffers were 20 mM phosphate, pH 11 (buffer A); and 20 mM phosphate, 1.8 M NaBr, pH 11 (buffer B). The flow rate 1.0 mL/min and monitored wavelength was 260-280 nm. Injections of 5-15 μL were done for each sample.

The unconjugated samples were purified by HPLC on a TSK-Gel SuperQ-5PW (20) column packed in house (17.3×5 cm) or on a commercially available TSK-Gel SuperQ-5PW column (15×0.215 cm) available from TOSOH Bioscience. The buffers were 20 mM phosphate in 10% CH₃CN, pH 8.5 (buffer A) and 20 mM phosphate, 1.0 M NaBr in 10% CH₃CN, pH 8.5 (buffer B). The flow rate was 50.0 mL/min for the in house packed column and 10.0 ml/min for the commercially obtained column. Wavelengths of 260 and 294 nm were monitored. The fractions containing the full-length oligonucleotides were pooled together, evaporated, and reconstituted to ˜100 mL with deionised water.

### (b) Cholesterol-Conjugated Oligonucleotides

The cholesterol-conjugated crude oligonucleotides were first analyzed by LC/MS to determine purity. The cholesterol conjugated sequences were HPLC purified on RPC-Source15 reverse-phase columns packed in house (17.3×5 cm or 15×2 cm). The buffers were 20 mM NaOAc in 10% CH₃CN (buffer A) and 20 mM NaOAc in 70% CH₃CN (buffer B). The flow rate was 50.0 mL/min for the 17.3×5 cm column and 12.0 ml/min for the 15×2 cm column. Wavelengths of 260 and 284 nm were monitored. The fractions containing the full-length oligonucleotides were pooled, evaporated, and reconstituted to 100 mL with deionised water.

Step 5. Desalting of Purified Oligonucleotides

The purified oligonucleotides were desalted on either an AKTA Explorer or an AKTA Prime system (Amersham Biosciences) using a Sephadex G-25 column packed in house. First, the column was washed with water at a flow rate of 40 mL/min for 20-30 min. The sample was then applied in 40-60 mL fractions. The eluted salt-free fractions were combined, dried, and reconstituted in ˜50 mL of RNase free water.

Step 6. Purity Analysis by Capillary Gel Electrophoresis (CGE), Ion-Exchange HPLC (IEX), and Electrospray LC/Ms

Approximately 0.3 OD of each of the desalted oligonucleotides were diluted in water to 300 μL and were analyzed by CGE, ion exchange HPLC, and LC/MS.

Step 7. Duplex Formation

For the fully double stranded duplexes, equal amounts, by weight, of two RNA strands were mixed together. The mixtures were frozen at −80° C. and dried under vacuum on a speed vac. Dried samples were then dissolved in 1×PBS to a final concentration of 40 mg/ml. The dissolved samples were heated to 95° C. for 5 min and slowly cooled to room temperature.

Step 8. Tm Determination

For the partial double stranded duplexes and hairpins melting temperatures were determined. For the duplexes, equimolar amounts of the two single stranded RNAs were mixed together. The mixtures were frozen at −80° C. and dried under vacuum on a speed vac. Dried samples were then dissolved in 1×PBS to a final concentration of 2.5 μM. The dissolved samples were heated to 95° C. for 5 min and slowly cooled to room temperature. Denaturation curves were acquired between 10-90° C. at 260 nm with temperature ramp of 0.5° C./min using a Beckman spectrophotometer fitted with a 6-sample thermostated cell block. The Tm was then determined using the 1st derivative method of the manufacturer's supplied program.

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A modified small activating ribonucleotide (saRNA) agent, wherein the saRNA agent is conjugated to a folate moiety.

2. An saRNA agent incorporated into a delivery vehicle, wherein the delivery vehicle is an association complex comprising one or more of the following: TETA-5LAP, or DLinDMA.

3. An saRNA agent incorporated into a delivery vehicle, wherein the delivery vehicle is an association complex comprising 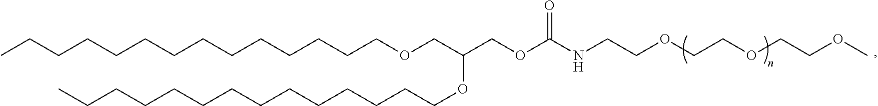
wherein n ranges from 0-100.

4. The saRNA of claim 3, wherein n ranges from 42 and 47.

5. The saRNA of claim 3, wherein the repeating polyethylene glycol (PEG) moiety has an average molecular weight of at least 2000.

6. The saRNA of claim 3, wherein the complex has an average molecular weight of at least 2000.
